(19) United States
(12) Patent Application Publication
(10) Pub. No.: US 2010/0119529 A1
(43) Pub. Date: May 13, 2010

(54) ELASTIN-LIKE POLYMER DELIVERY VEHICLES

(76) Inventors: Darin Y. Furgeson, Salt Lake City, UT (US); Younsoo Bae, Lexington, KY (US); Glen S. Kwon, Waunakee, WI (US)

Correspondence Address: FULBRIGHT & JAWORSKI L.L.P., 600 CONGRESS AVE., SUITE 2400, AUSTIN, TX, 78701, US

(21) Appl. No.: 12/299,890

(22) PCT Filed: May 11, 2007
(86) PCT No.: PCT/US07/68800

§ 371 Date: Jun. 12, 2009

Related U.S. Application Data
(60) Provisional application No. 60/799,798, filed on May 12, 2006.
(60) Provisional application No. 60/832,455, filed on Jul. 21, 2006.
(60) Provisional application No. 60/864,919, filed on Nov. 8, 2006.

(51) Int. Cl. A61K 39/395; C07K 14/00; C07K 17/02; A61P 35/00; A61P 9/00; A61P 25/00

(52) U.S. Cl. 424/181.1; 530/350; 530/391.1

(57) ABSTRACT

In invention concerns elastin-like polymer (ELP) drug delivery compositions and methods for the use thereof. In some aspects ELP delivery vehicles may be used to deliver therapeutic drugs such as Hsp90 antagonists. Furthermore, embodiments of the invention concern in vivo delivery with ELP compositions directed to target sites by the application of local hyperthermia therapy. Methods of the invention may have particular utility in the delivery of geldanamycin and related drugs.

This application claims priority to U.S. provisional patent application Ser. No. 60/799,798, filed May 12, 2006; U.S. provisional patent application Ser. No. 60/832,455, filed Jul. 21, 2006; and U.S. provisional patent application Ser. No. 60/864,919, filed Nov. 8, 2006, each incorporated by reference in their entirety.

This invention was made with government support under AI043346 awarded by the U.S. National Institute of Health. The government has certain rights in the invention.

### BACKGROUND OF THE INVENTION

I. Field of the Invention

The invention generally concerns the fields of medicine and molecular biology. In particular, the invention concerns polypeptides for delivery of therapeutic molecules method for the use thereof.

II. Description of Related Art

In the past, therapeutic drugs have generally been delivered by passive or nonspecific targeting. Passive targeting includes targeting based upon size, ionic state, and biological factors and is limited the ability of the therapeutic to diffuse to its site action and the rate of clearance for the therapeutic. Intravenously injected molecules, for example, may have to traverse a cell membrane to reach a site of action and may be readily processed or degraded by the body, thus limiting their use and efficacy. Additionally, the hydrophobicity of some therapeutic molecules has proven problematic since therapeutically effective concentrations of such molecules are difficult to disperse in a subject. To address these issues, synthetic polymers such as poly(N-isopropylacrylamide) (Schild, 1992), poly(ethylene glycol)-block-poly(caprolactone) copolymers (Kim et al., 2004), poly(ethylene oxide)-poly(propylene oxide) multiblock copolymers (Sosnik and Cohn, 2005) and multiple hydrogen bonding-poly(butylenes terephthalate) (Yamauchi et al., 2004) have been used as monolithic gels to deliver drugs. Unfortunately, synthetic polymers such as these suffer from the effects of polydispersity, lack of architectural control, variable levels of biocompatibility and complex synthesis schemes. In particular, traditional synthetic polymers lack this degree of molecular weight control; therefore, there is significant promise for the use of genetically engineered polymers in gene and drug delivery (Kopecek, 2003).

Thus, on merely a biophysical basis, genetically engineered biopolymers such as elastin-like polymers (ELP) pose an attractive alternative to traditional polymer macromolecules for gene and drug delivery due to their monodispersity, non-immunogenicity, and unparalleled control of architecture and biophysical characteristics (“smart” polymer behaviors, ionization state, hydrophobicity). Genetic engineering confers precise control of the biophysical characteristics of biopolymers, a level of control yet to be realized in synthetic polymer syntheses. Through molecular biology techniques, the pentapeptide sequence, molecular weight, and architecture of the ELP can be precisely controlled for subsequent an purified as a recombinant polypeptide, resulting in monodisperse, non-immunogenic (Urry et al., 1991) ELP biopolymers with variable ionic and hydrophobicity characteristics.

Genetically engineered biopolymers show great promise as macromolecule, gene and drug carriers due to genetic control of composition and monodispersity. Moreover, elastin-like polymers are thermosensitive enabling methods for hyperthermic targeting to specific sites for therapy. The use of ELP-biomacromolecules and ELP-biopolymers for the delivery of certain drugs (Dreher et al., 2003; Herrero-Vanrell et al., 2005) and peptides (Bidwell and Raucher, 2005) has been reported. However, previously there has not been an effective ELP platform that could be used to deliver the array of therapies currently in use in the medical field.

### SUMMARY OF THE INVENTION

The instant invention overcomes deficiencies in the prior art by providing a polypeptide delivery vehicle for therapeutic compositions. Polypeptide delivery vehicles of the invention generally comprise an elastin-like polypeptide (ELP) in complex with a therapeutic molecule. For example, such an ELP composition may comprise an ELP complexed with a therapeutic small molecule, polypeptide or nucleic acid. In some cases, an ELP may be covalently linked to a therapeutic molecule. For instance, an ELP composition may comprise an ELP domain covalently linked to a small molecule or an ELP linked to a therapeutic polypeptide by a peptide bond (i.e., an ELP fusion protein). In some further cases, an ELP may be fused with a polypeptide that binds to a therapeutic molecule. Likewise, an ELP may be in complex with or covalently conjugated to a nucleic acid aptamer, such as an aptamer that binds to a therapeutic molecule.

Thus, in some aspects, the invention provides a drug delivery vehicle comprising a therapeutic drug complexed with an elastin-like polypeptide. An ELP may be covalently or non-covalently complexed with a small molecule drug. For example, a drug may be chemically conjugated to an ELP domain or conjugated to an additional polypeptide domain in an ELP composition. For instance, an ELP composition may be conjugated to cisplatin, geldanamycin or doxorubicin. In some further embodiments, an ELP composition may comprise a polypeptide domain that specifically binds to a small molecule. As described in detail below, drug delivery vehicles comprising small molecules may further comprise additional polypeptide domains, such as a nucleic acid binding or therapeutic polypeptide domain, or additional elements in complex with the delivery vehicle such as nanoparticles or nucleic acids.

Thus, certain aspects, a drug delivery vehicle of the invention may comprise a Hsp90 antagonist complexed with an elastin-like polypeptide. An hsp-90 antagonist is defined herein is a small molecule that binds to Hsp90 and antagonizes its activity. For example an Hsp90 antagonist may be a benzoquinoid ansamycin, radicicol, or a derivative thereof. Some exemplary benzoquinoid ansamycins include but are not limited to geldanamycin (GA), 17-allylamino-17-demethoxygeldanamycin (17-AAG), 17-(dimethylaminoethylamino)-17-demethoxygeldanamycin and 17-dimethylaminoethylamino-17-demethoxy-geldanamycin (17-DMAG) (Smith et al., 2005). Furthermore, any of the GA derivatives described in the U.S. patent application entitled “Micelle Composition of Polymer and Passenger Drug,” may be used according to the current invention (U.S. application Ser. No. 11/402,639, incorporated herein by reference). The term Hsp90 antagonist also encompasses prodrugs or drugs that upon metabolic processing in an animal gain Hsp90 antagonist activity. In some aspects, an Hsp90 antagonist may be covalently conjugated to an ELP domain. For example, an Hsp90 antagonist may be conjugated to an ELP domain via a cleavable linker, such as a pH cleavable linker, a photo-sensitive linker, an enzyme cleavable linker, a heat cleavable linker, a radiation cleavable linker or a linker that is cleaved in aqueous solution. In some specific aspects, a linker may be a water or enzyme cleavable ester linkage as exemplified herein.

As used herein the terms “elastin-like polypeptide” or “elastin-like repeat” (ELP) are used interchangeably. ELP refers to a class of amino acid polymers that undergo a conformation change dependent upon temperature. By increasing the temperature ELPs transition from elongated chains that are highly soluble into tightly folded aggregates with greatly reduced solubility (see U.S. Pat. No. 6,852,834). An ELP may, for example, be defined by the median temperature at which this phase transition occur. Thus, in certain aspects of the invention, an ELP will have a median phase transition temperature above about 37° C. In some further embodiments, an ELP may have a median phase transition temperature in a physiological range such as a transition temperature of about 38° C., 39° C., 40° C., 41° C., 42° C., 43° C., 44° C., 45° C. or 46° C. In some cases, ELPs may also be defined based upon the temperature range over which the phase transition occurs. For example, in some cases, the phase transition will occur over a temperature range of less than about 5° C. For instance phase transition may occur in a temperature range of about 4° C., 3° C., 2° C., 1° C. or less.

In some specific embodiments of the invention, an ELP domain may be defined by its amino acid sequence. For example, an ELP domain may comprise multiple repeats of the amino acid sequence VPGXG, wherein X is any amino acid except proline. For example, an ELP of the invention may comprise 10 to 500 repeats of the VPGXG sequence. In some even more specific cases, an ELP of the invention may comprise between 50 and 300 or 80 and 200 amino acids. In some embodiments, the “X” residues in an ELP will all be the same amino acid, however certain other cases an ELP may comprise a variety different residues in the X position throughout the polymer. For example, in some cases X may be an alanine, a valine or a glycine residue, such as an ELP that comprises 10 VPGXG repeats wherein X=Val for the first five repeats, X=Ala for the next two repeats and X=Gly for the remaining 3 repeats (denoted V₅:A₂:G₃).

As further discussed in the detailed embodiments, the sequence of an ELP may be further modified by amino acid substitutions deletion or insertions. Such changes in the ELP amino acid sequence may be used, for example, in order to adjust the median phase transition temperature or the range at which phase transition occurs. For example, an ELP may be defined as amphipathic ELP comprising an ELP domain that is highly hydrophobic and a second ELP domain that is more hydrophilic. In some instances an amphipathic ELP could be generated by substituting different hydrophobic or hydrophilic amino acids at the X for two different ELP domains. An amphipathic ELP would be expected to exhibit a biphasic temperature transition profile since the hydrophobic ELP domains would begin to aggregate at a lower temperature than the hydrophilic ELP domain. In some cases, the hydrophobic domain of an amphipathic ELP complex may be defined as having a transition temperature that is below animal physiological temperatures (e.g., 37° C.) or below room temperature, while the hydrophilic ELP domain may be defined as having a transition temperature above 37° C., such as transition temperature between about 39° C. and about 45° C.

In further embodiments, a drug delivery vehicle that comprises a therapeutic drug (e.g., an Hsp90 antagonist) in complex with an ELP may be defined by the median phase transition temperature of the complex. For instance, a drug delivery vehicle may have median phase transition temperature above about 37° C. Furthermore, in some cases a drug delivery vehicle may have a median phase transition temperature in a physiologically relevant range such as a transition temperature of about 38° C., 39° C., 40° C., 41° C., 42° C., 43° C., 44° C., 45° C. or 46° C. Additionally, drug delivery vehicles of the invention may be defined based upon the temperature range over which the phase transition occurs. For example, in some cases, the phase transition will occur over a temperature range of less than about 10° C. or less than about 5° C.

In still further aspects of the invention, a drug delivery vehicle may be defined by the median diameter of the complex. For instance, a drug delivery vehicle may have a median diameter of less than about 1 μm. In yet more specific instances, a drug delivery vehicle may be defined as having a median diameter of about or less than about 500, 400, 300, 200, 100 or 50 nM. Thus, in certain cases, a plurality of drug delivery vehicles may be defined by the average median diameter within the plurality of drug delivery vehicles (e.g., the plurality of drug deliver vehicles may have an average median diameter of less than about 1 μm). Thus, in certain aspects of the invention it is contemplated that drug delivery vehicles may be provided as dimers, trimers or higher order complexes of individual ELP molecules.

As discussed supra, in some aspects a drug delivery vehicle may comprise a Hsp90 antagonist non-covalently complexed with an ELP domain. A drug (e.g., GA) may, for instance, be encapsulated by ELP complex. Thus, in some specific cases amphipathic ELPs may be used to encapsulation drugs by mixing ELPs and drug at a low temperature than raising the temperature past the transition temperature of the hydrophobic ELP domain. Aggregated hydrophobic domains will entrap drugs, especially hydrophobic drugs such as GA, whereas the hydrophilic ELP portion allows the drug ELP complexes to remain relatively soluble. Drug encapsulation efficiency and ELP particle size may be modulated by adjusting a variety of factors including, but not limited to, the rate of temperature increase, the pH of the solution, the concentrations of drug or ELP, the amount of agitation applied or by the addition of agents that alter the solubility of drugs or ELPs such as glycerol or DMSO.

Thus, in still a further embodiment, there is provided an Hsp70 antagonist micelle composition. For example, a micelle composition may comprises an Hsp70 antagonist such as GA or a GA derivative conjugated to or in complex with a hydrophilic polymer. In certain aspects, the hydrophilic polymer may be an ELP, a polyethylene glycol (PEG) or hydrophilic amino acid sequence such as polyaspartic acid or a poly lysine sequence (e.g., a block polymer). Thus, in this aspect, the Hsp70 antagonist may form the hydrophobic core of the micelle while the hydrophilic polymer forms the micelle corona. As described elsewhere, in some cases drug conjugation may be though a water cleavable ester and thus the linkage would be partially protected by the micelle structure. Furthermore, such micelle formulations may be conjugated to additional functional elements such as a cell targeting moiety, a nucleic acid binding domain or an energy absorbing nanoparticle.

In some further embodiments of the invention, a drug delivery vehicle may be provided in a complex with a particle that absorbs energy and is thereby heated. Such particles (e.g., nanoparticles) may be complexed with drug delivery vehicles non-covalently as previous described or may be covalently conjugated to a delivery vehicle. For example, particles may absorb radio frequency radiation or be heated by magnetic induction as described in U.S. Publn. Nos. 20050251234 and 20050090732. In some aspects, particles for use in the invention may be defined as metal nanoparticles or metal nanoshells.

In further embodiments of the invention, an ELP may comprise an ELP domain and a nucleic acid binding moiety. In certain cases, the nucleic acid binding moiety may be conjugated to the ELP, for example via a covalent chemical conjugation. However, in some other cases, the nucleic acid binding moiety is a polypeptide and the ELP composition may be a fusion protein comprising an ELP domain and the nucleic acid binding domain. Any nucleic acid binding polypeptide know in the art may be used for an ELP composition of the invention. For example, a nucleic acid binding domain may bind to a specific nucleic acid sequence, such as the RNA binding domains of iron regulatory protein (IRP) 1 or 2. In certain additional cases, the nucleic acid binding sequence may bind to nucleic acids non-specifically, such as amino acid polymers that are rich in cationic residues. For example, a nucleic acid binding domain may have of 25%, 30%, 35%, 40%, 45%, 50% or more residues that are positively charged at physiological pH, such as lysine. In certain instances, a nucleic acid binding polypeptide may comprise repeats of the amino acid sequence VK or VKG. For instance, the sequence may have 4 to 100 VK or VKG repeats or a mixture thereof, such as nucleic acid binding domain with 4, 8, 12, 16, 20, 24, 28, 32, 36, 40, 44, 48, 52, 56, 60, 64, 68, 72, 76, 80, 84, 88, 92, 96 or 100 VK or VKG repeats. Thus, a drug such as GA may be complexed with a nucleic acid binging domain of an ELP.

In yet further embodiments of the invention, a drug delivery vehicle may comprise a cell targeting domain, such as an antibody, ligand or nucleic acid aptamer. Such a domain may be conjugated to an ELP composition of the invention. In certain cases, an ELP composition may be a fusion protein comprising an ELP domain and a cell targeting domain, and it yet further cases such a fusion protein may also comprise a nucleic acid binding domain. A cell targeting domain may, for example, be an antibody, a ligand, a cytokine or a chemokine. Cell targeting antibodies include, but are not limited to, polyclonal antibodies, monoclonal antibodies, single chain antibodies, antibody fragments, or humanized antibodies. In another example, a cell targeting ligand may be VEGF or the amino acids there from that mediate receptor binding. Cell targeting domains may preferentially bind to certain classes of cells such as immune cells, cancer cells, or cells from a particular tissue or lineage. In certain aspects of the invention, cell targeting moieties may also mediate internalization an ELP composition.

In still further embodiments of the invention, a drug delivery vehicle of the composition may comprise a membrane translocation or cell localization domain. Such a domain may be conjugated to an ELP composition of the invention. Furthermore, an ELP composition may be a fusion protein comprising an ELP domain and membrane translocation domain or cell localization domain. Such a fusion protein, in some cases, may also comprise a nucleic acid binding domain. For example, an ELP composition may comprise a membrane translocation domain such as amino acids for the HIV tat protein or the drosophila antennapedia protein. In some additional aspects, an ELP composition comprises a cell localization domain, such as a nuclear localization signal, a secretion signal, an endoplasmic reticulum retention signal, a lysosome localization signal or a mitochondrial localization signal. Thus, in certain specific cases, a drug delivery vehicle may comprise an ELP domain conjugated to a drug via an acid labile linker and a membrane translocation or cell localization domain that will mediate transport of the complex into a lysosome, thereby enabling releasing the drug upon lysosome acidification.

In still further embodiments of the invention, a drug delivery vehicle further comprising a therapeutic polypeptide domain is provided. In certain aspects of the invention, a therapeutic polypeptide domain may be chemically conjugated to an ELP domain, however in certain cases the ELP and therapeutic polypeptide domains may be comprised in a fusion protein. Therapeutic polypeptides for use the instant invention include, but are not limited to, cytokines, chemokines, angiogenic factors, anti-angiogenic factors. In some specific examples, a therapeutic polypeptide may be interferon (IFN)-α, IFN-β, IFN-γ, IFN-τ, tumor necrosis factor (TNF)-α, HIF-1α, vascular endothelial growth factor (VEGF), fibroblast growth factor (FGF), platelet derived growth factor (PDGF), NF-κB inhibiting sequences, consensus interferon sequences, interleukin (IL)-2), IL-12, IL-4, IL-8 or a single chain antibody sequence (scFv) such as a VEGF specific scFv. In certain aspects of the invention, a therapeutic polypeptide may comprise an extra cellular domain of a receptor protein, such as VEGF receptor (VEGFR)-1 (Flt-1), VEGFR-2 (Flk-1/KDR) or VEGFR-3.

In some additional embodiments, an ELP composition may comprise a spacer or linker domain. A spacer domain may, for example, be positioned between any two domains in an ELP composition fusion protein. A spacer domain of the invention can comprise any number of amino acids and may include a variety of amino acid residues. For instance, a spacer region may comprise 3 or more histidine residues. Such a polyhistidine region can act as a pH buffer in an ELP composition. In some additional cases, spacer regions may comprise amino acids that are sensitive to proteinase cleavage, for example, proteinase cleavage induced by heat, cell stress or pH (e.g. low pH). In certain very specific cases, a proteinase sensitive sequence may be sensitive to an intracellular proteinase, an extracellular proteinase or a proteinase that is associated with metastasis of cancers such as matrix metalloproteinase (MMP).

It will be understood by one of skill in the art that domains of an ELP composition may be positioned in a variety orientations relative to one another. For example, in the case where an ELP composition is a fusion protein the ELP domain may be positioned near the amino terminus, near the carboxyl terminus or in the middle of the fusion protein. In certain aspects of the invention a an ELP composition fusion protein may comprise, from amino terminus to carboxyl terminus, a DNA binding domain, optionally a spacer domain, an ELP domain, optionally a linker domain and optionally a cell targeting domain or a membrane translocation domain.

In some embodiments, an ELP domain of a drug delivery vehicle is a fusion protein. Thus, there is provided a nucleic acid that encodes an ELP domain and fusion proteins thereof. Such a nucleic acid comprising the coding sequence for an ELP composition may also include addition sequences. For, example sequence for prokaryotic or eukaryotic expression of an ELP composition may be provided. Thus, included as part of the invention is a method for making an ELP composition, for example by expressing a nucleic acid encode the ELP composition in cell, such as bacterial cell. It will also be understood that the thermal transition properties of the ELP domain may be employed to aid in the purification of such ELP compositions (see for example, U.S. Pat. No. 6,852,834).

In some further aspects of the invention, there is provided a drug delivery vehicle comprising a therapeutic drug in complex with an ELP domain and a nucleic acid binding domain wherein the nucleic acid binding domain is complexed with a nucleic acid. The complex of such a drug delivery vehicle and a nucleic acid is herein termed a “bioplex.” For example, a bioplex of the invention may comprise an ELP composition, such as a fusion protein comprising an ELP domain and a nucleic acid binding domain complexed with a therapeutic nucleic acid. A bioplex of the invention may comprise a DNA or RNA molecule. For example, nucleic acids that may be used in a bioplex of the invention include, but are not limited to, DNA expression vectors, RNA expression vectors, siRNAs, miRNAs, aptamers and ribozymes. Bioplex nucleic acids may in some cases be therapeutic nucleic acids that may be used in gene therapy. For example, a therapeutic nucleic acid may induce apoptosis in cancer cells or restore the function a mutant gene to correct a genetic disorder. It will be understood by the skilled artisan that in some aspects of the invention a bioplex that comprises bound nucleic acid will release at least 20, 30 40, 50, 60, 70, 80, 90, 95 percent or more of the bound nucleic acid at temperatures above the transition temperature for the bioplex.

In yet further aspects of the invention, a bioplex may be defined by the ratio of ELP composition to nucleic acid in the bioplex. In some aspects, the ELP composition to nucleic acid ratio may be defined as a simple molar ratio. However, in further cases, a ratio may be defined as the ratio of the amino acid nitrogens (in a nucleic acid binding domain) to nucleic acid phosphates (N/P). Thus, in certain aspects of the invention, the N/P ration may be between about 50 to 1 (50/1) and about 1 to 1 (1/1). In still further aspects, a bioplex may have an N/P ration of about 50/1, 45/1, 40/1, 35/1, 30/1, 25/1, 20/1, 15/1, 10/1, 9/1, 8/1, 7/1, 6/1, 5/1, 4/1, 3/1, 2/1 or 1/1 or any range derivable therein.

In still further embodiments, there is provided a method for making a bioplex drug delivery vehicle comprising mixing an ELP composition with a nucleic acid molecule. Furthermore, there is provided a method for delivery of a therapeutic nucleic acid to a cell comprising, mixing the therapeutic nucleic acid with an ELP composition (comprising an ELP and a nucleotide binding sequence) to form a bioplex and contacting the cell with the bioplex. In certain aspects, the ELP composition may be further defined as a fusion protein comprising at least ELP domain and a nucleic acid binding domain. In some cases, it will be understood that the soluble bioplex may be used to transfect cells with a nucleic acid. However, in certain aspects of the invention, a bioplex may be transitioned into an insoluble form in order to transfect a cell. A bioplex may be transitioned into an insoluble form (i.e., an aggregate) either before or after contacting a cell with the bioplex. In some specific cases, a bioplex maybe transitioned into an insoluble form by the application of heat (i.e., by increasing the temperature of the bioplex).

Certain aspects of the invention concern methods for delivery of a drug to a cell by contacting the cell with a drug delivery vehicle or a bioplex. It will be understood that such methods may comprise in vitro, ex vivo or in vivo nucleic acid delivery. Thus, in some cases, a drug delivery vehicle of the invention may be administered to a human.

In still further embodiments, there is provided a method for treating a disease in an animal comprising administering to the animal and effective amount of a drug delivery vehicle of the invention. Such methods may employ any of the drug delivery vehicles described herein. It will also be understood that methods and compositions of the invention may be adapted to treat a variety of diseases including but not limited to a wound, a cardiovascular disease, an infection (e.g., a viral infection), a genetic disorder, a protein aggregation disease, an autoimmune disease or a cell proliferative disease such as cancer. For example, drug delivery vehicles may be used in the treatment of a protein aggregation disease such as Huntington's disease, Alzheimer's disease, Parkinson's disease, or a prion disease. Furthermore, in certain aspects, viral diseases such as hepatitis B or C infections may also be treated by methods of the invention.

In some aspects, methods for treating a cell proliferative disease are provided by the instant invention. Such cell proliferative diseases include, but are not limited to, cancers and angiogenic disorders. For example, an angiogenic disorder may be treated ocular neovascularization, Arterio-venous malformations, coronary restenosis, peripheral vessel restenosis, glomerulonephritis or rheumatoid arthritis. In some specific cases, methods of the invention also concern the treatment of a cancer such as a melanoma, non-small cell lung, small-cell lung, lung, hepatocarcinoma, retinoblastoma, astrocytoma, glioblastoma, gum, tongue, leukemia, neuroblastoma, head, neck, breast, pancreatic, prostate, renal, bone, testicular, ovarian, mesothelioma, cervical, gastrointestinal, lymphoma, brain, colon, sarcoma or bladder cancer. Thus, is some very specific embodiments, there are provided methods for treating a tumor with a drug delivery vehicle of the invention.

Drug delivery vehicles of the invention may be administered by a variety of routes including, but not limited to intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, locally, by inhalation (e.g., aerosol inhalation), by injection, by infusion or by continuous infusion. Thus, compositions of the invention may be delivered locally or systemically.

In some aspects the invention provides a method for slow or controlled release of a therapeutic molecule. For example, an ELP delivery vehicle may comprise a transition temperature near or below a physiological temperature. Thus, at room temperature, a delivery vehicle may be soluble in aqueous solution while aggregating when exposed to higher temperatures (e.g., normal body temperature). Thus, in some aspects an ELP delivery vehicle rapidly upon administration to an animal. The insoluble aggregate may be maintained in the animal over a period of time an as it degrades a therapeutic molecule may be released thereby providing an extended or slow release of platform for therapeutic molecules. Similar methods have bee described in the case of certain synthetic polymers, such as those comprising the REGEL® product (U.S. Pat. Nos. 6,201,072, 6,117,949, 6,004,573 and 5,702,717). Advantageously, an ELP delivery vehicle is completely bio compatible and thus would not elicit an adverse immune response. Furthermore, degradation of ELP aggregates will occur through normal protein clearance pathways in the animal and thus would not result in toxic breakdown products.

Furthermore, it will be understood that methods of treating a disease with a drug delivery vehicle may be used in combination or in conjunction with additional therapies such as hyperthermia, chemotherapy, surgical therapy, radiotherapy, immunotherapy, or gene therapy. Such additional therapies may be employed before, after or concomitantly with the administration of a drug delivery vehicle.

Thus, in certain embodiments, administration of a drug delivery vehicle may be used in combination with hyperthermia (heat therapy). Hyperthermia may be applied before, after or essentially simultaneously with the administration of a drug delivery vehicle. In some aspects of the invention, hyperthermia may be applied to the whole body of an animal. However, in some other cases, hyperthermia may be applied locally. For example, hyperthermia may be applied only to a specific region of the body such as a wound, an organ, a site of infection or a tumor. It will be understood that hyperthermia therapy may comprise increasing the temperature of a region to any temperature that is above that of a normal, healthy, animal. For example, in the case of a human hyperthermia therapies may comprise raising the temperature of region to above about 37° C. Furthermore, the temperature of a region may be raised to between about 38° C. and about 46° C. In some very specific cases, the temperature may be raised to about or at least about 38° C., 39° C., 40° C., 41° C., 42° C., 43° C., 44° C., or 45° C.

Methods for applying hyperthermia are well known in the art. For example, for example tissues or cells may be directly heated using a heated surface or probe, or in some cases may be heated with microwave radiation or ultrasonic waves. In other aspects, hyperthermia may be applied indirectly using radio frequency radiation or magnetic induction to mediate heating of particles that absorb these forms of energy (see for example U.S. Pubin. Nos. 20050251234 and 20050090732). In this aspect, methods of the invention may further comprise the administration of absorbing particles to an animal or the incorporation such particles into drug delivery vehicles thereby enabling indirect hyperthermia therapy.

In certain specific aspects, a hyperthermia temperature may be raised to a temperature that is above the transition temperature a drug delivery vehicle. For instance, following administration of a drug delivery vehicle hyperthermia may be applied to one or more locations wherein the temperature at the application site is about 0.5° C., 1.0° C., 1.5° C., 2.0° C., 2.5° C., 5° C., 10° C. or greater than the median transition temperature of the delivery vehicle thereby enabling maximal drug delivery vehicle aggregation at the site(s). Furthermore, hyperthermia may be applied in two or more cycles thereby allowing drug delivery vehicle aggregation over an extend time period. In still further aspects of the invention, hyperthermia may be applied to one or more sites wherein the temperature at the application site reaches a temperate about 1.5° C., 1.0° C., 0.5° C. or less above or below the transition temperature of a drug delivery vehicle thereby allowing slow aggregation and/or accumulation at the site. Thus, in certain aspects the invention provides a method for dosed delivery of a drug to a site of hyperthermia. Additionally, in certain aspects, the invention provides methods for localized drug delivery in an animal by locally or systemically administering a drug delivery vehicles and locally administering hyperthermia therapy.

Furthermore, amphipathic ELP-drug complexes may be used in the treatment methods of the invention. In some cases, these complexes may be used in conjugation with hyperthermia therapy. In such cases it will be understood that hyperthermia therapy may involving raising the temperature of an animal or localized region of an animal to a temperature higher than the median transition temperature of hydrophilic ELP domain. As discussed herein, in some cases amphipathic ELPs may be used to non-covalently encapsulate therapeutic drugs. In these aspects, hyperthermia may be used not only to target delivery vehicle accumulation at a therapy site, but also may result in a rearrangement of the ELP-drug complex resulting in enhanced drug release at the site of action (i.e., the site of hyperthermia). Thus, in certain aspects the invention provides methods for targeted drug release in an animal.

Embodiments discussed in the context of a methods and/or composition of the invention may be employed with respect to any other method or composition described in this application. Thus, an embodiment pertaining to one method or composition may be applied to other methods and compositions of the invention as well.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings are part of the present specification and are included to further demonstrate certain aspects of the present invention. The invention may be better understood by reference to the drawings in combination with the detailed description of specific embodiments presented herein.

FIG. 1. An example conjugation scheme for geldanamycin-(VKG)₈ELP conjugate. Reaction condition: (i) 2-ethylamine/CHCl3/RT/2 hr, (ii) succinic anhydride/CHCl3/TEA/RT/4 hr, (iii) DCC/DMSO/RT/5 hr.

FIG. 2. Determination of GA-ELP conjugation efficiency. UV-visible spectra of native (triangles) and GA-conjugated (VKG)₈ELP1-120 (squares) at 70 μM concentration (ELP-based).

FIG. 3. Profiles of the thermal transition of native (25 μM) and GA-(VKG)₈ELP1-120 conjugate as a function of temperature. The concentration of the GA-(VKG)₈ELP1-120 conjugates varied from 3.125 to 25 μM normalized to the elastin block.

FIG. 4. Synthesis scheme of [K8-ELP(1-60)]-GA conjugates: 1: geldanamycin (GA); 2: 17-aldehyde-geldanamycin [GA(CHO)]; 3: 17-hydroxyethyl-geldanamycin [GA(OH)]; 4: 17-(ethylamino-succinate)-geldanamycin; 5: [K8-ELP(1-60)]-GA(CHO)conjugate; and 6: [K8-ELP(1-60)]-GA(OH)conjugate.

FIG. 5. UV-Vis (inset) and 1H-NMR analysis to determine drug conjugation ratio.

FIG. 6. Thermosensitive phase transition profiles of native K8-ELP(1-60) block copolymers and [K8-ELP(1-60)]-GA(CHO) conjugates at various concentrations.

FIG. 7. Particle size measurements at different temperatures (25 and 80° C.). Data were obtained from triplicate measurements. The values for particle size are shown as mean±SD.

FIG. 8. In vitro cytotoxicity of parent geldanamycin (GA), geldanamycin derivative [GA(CHO)], block copolymers [K8-ELP(1-60)], and geldanamycin-polymer conjugates [[K8-ELP(1-60)]-GA(CHO)] against human breast cancer MCF-7 cells at normothermic (NT) and hyperthermic (HT) conditions.

FIG. 9. Comparison between the IC₅₀ values obtained from in vitro cytotoxicity experiments. Data are regenerated to calculate the relative index, which shows an increase in cytotoxic activity of the samples compared to the activity of parent geldanamycin.

### DETAILED DESCRIPTION OF THE INVENTION

Successful in vivo delivery of therapeutic compositions such as drugs involves a number obstacles. For example, in vivo therapeutics must have a long enough serum half life to be effective, be soluble at effective concentrations and be able reach the site of therapeutic action. Heat shock protein 90 (Hsp90) antagonist drugs, despite their promise as chemotherapeutic agents, have proven difficult to effectively deliver in vivo. For example, geldanamycin (GA) analogs, have shown great promise as a potential therapeutic for a variety of disease, but also has many drawbacks related to effective delivery such as low aqueous solubility (Sharp and Workman, 2006). Furthermore, the many effective drugs also have significant toxicity, and thus methods for targeted delivery would be preferred in order to reduce toxicity (Glaze et al., 2005). Previously, there have not been completely effective therapeutic delivery vehicles for use in delivering and targeting of drugs such as GA.

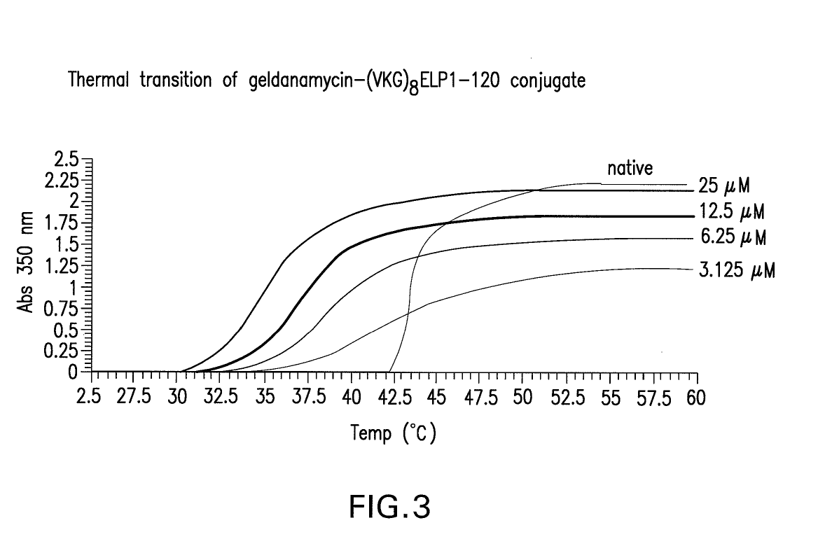

The studies herein demonstrate that elastin-like polypeptides can be used as effective drug delivery vehicles. Experimental results here show that ELP domains can be conjugated to an Hsp90 antagonist such as a GA analog. Drug conjugation is demonstrate to be efficient and importantly ELP-drug complexes maintain the key thermal transition properties of the ELP domain (see FIG. 3). Furthermore, ELP-drug complexes remain highly cytotoxic to cancer cells indicating that the therapeutic properties of the drug are not significantly hampered by the ELP domain. Thus, these studies indicate that ELP delivery vehicles may be used for improved drug delivery and targeting thereby enabling new methods for administration of therapeutic molecules.

Thus, the invention sets forth a unique approach to therapeutic drug delivery by providing a biopolymer delivery composition. The composition is highly soluble in aqueous solutions, but can be caused to aggregate along with its therapeutic payload, by increasing the local temperature. Thus, delivery vehicles described herein may be introduced systemically in a patient and, in cases where a local therapy is preferable, the temperature in a localized region may be raised thereby resulting in accumulation of the delivery vehicle at the therapy site. Delivery vehicles of the invention may be of particular interest for delivery of GA or GA analogs since the delivery vehicles offer a soluble and targeted delivery platform for GA therapy. These advantages address the major problem associated with GA, aqueous insolubility and toxicity effects. Furthermore, hyperthermia targeted delivery methods may act to further enhance the therapeutic efficacy of Hsp90 antagonist drugs by inducing heat stress response in targeted cells thereby enhancing the cytotoxicity of Hsp90 antagonist molecules. Thus, methods and compositions described herein represent a significant advance in the field of drug delivery and targeted drug therapy.

### I. NUCLEIC ACIDS

The present invention concerns a number of different types of nucleic acid molecules that can be used in a variety of ways. In some embodiments of the invention, the nucleic acid is a recombinant nucleic acid. The term “recombinant” is used according to its ordinary and plain meaning to refer to the product of recombinant DNA technology, e.g., genetically engineered DNA prepared in vitro by cutting up DNA molecules and splicing together specific DNA fragments, which may or may not be from different organisms. Things that have or are from a genetically engineered DNA are similarly recombinant; this includes replicated or duplicated products based on the initially engineered DNA. In particular embodiments, the invention concerns therapeutic nucleic acids recombinant DNA and RNA molecules.

In some embodiments, the nucleic acid molecule is a DNA molecule, for example, a DNA molecule whose expression gives rise to the RNA transcript. Alternatively, the DNA molecule may be used in a protein expression (e.g., ELP compositions) or a therapeutic method of the invention or the molecule may encode an RNA transcript or polypeptide that is used in such methods. These different DNA molecules may or may not be in an expression construct such as a vector or in a host cell. Further details are provided below.

The present invention concerns polynucleotides, isolatable from cells, that are free from total genomic DNA and that are capable of expressing all or part of an RNA molecule, RNA transcript, protein or polypeptide. The polynucleotide may be an RNA molecule such as an siRNA, an miRNA or a ribozyme. Alternatively, a polynucleotide may encode a peptide or polypeptide having all or part of the amino acid sequence of a therapeutic protein.

Embodiments of the invention concern isolated and/or recombinant polynucleotides. An isolated polynucleotide refers to a polynucleotide that is separated from a cell and its non-nucleic acid contents, and more specifically, may be separated from other nucleic acid sequences. A recombinant polynucleotide refers to a genetically engineered nucleic acid molecule or products of such a molecule (either through duplication, replication, or expression).

As used in this application, the term “transcript” refers to a ribonucleic acid molecule (RNA) that in some embodiments of the invention is generated from a recombinant DNA molecule. In particular embodiments, polynucleotides of the invention concern transcripts that encode ELP compositions or that may be used as a gene therapy therapeutic.

The term “cDNA” is intended to refer to DNA prepared using messenger RNA (mRNA) as template. In many embodiments of the invention, the nucleic acid is a cDNA or cDNA sequence. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

It also is contemplated that a particular RNA molecule or transcript from a given species may be represented by natural variants that have slightly different nucleic acid sequences but, nonetheless, encode what is considered a wild-type sequence.

It is contemplated that nucleic acid molecules encoding RNA molecules with a nucleotide repeat region may be used in method and compositions of the invention. Furthermore, candidate substances or compounds, candidate therapeutic agents, or other agents may be employed as nucleic acids, including recombinant nucleic acids in compositions and methods of the invention.

In other embodiments, the invention concerns isolated nucleic acid molecules and recombinant vectors incorporating DNA sequences that encode a polypeptide or peptide that includes within its amino acid sequence a contiguous amino acid sequence in accordance with, or essentially corresponding to the polypeptide.

The nucleic acid segments used in the present invention, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

It is contemplated that the nucleic acid constructs of the present invention may encode part or all (full-length) of transcripts or polypeptides from any source. Alternatively, a nucleic acid sequence may encode an RNA or polypeptide with additional heterologous sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a sequence that is not the same from the same source as other sequences.

In certain other embodiments, the invention concerns isolated DNA or RNA segments and recombinant vectors that include within their sequence the coding sequence for an ELP composition or a therapeutic protein. One of skill in the art will understand the due to the degeneracy of the genetic code a variety of nucleic acid sequence can encode a single amino acid sequence (see for instance the codons listed in Table 1). Therefore, it is contemplated that any nucleic acid sequence capable of encoding a polypeptide of the invention is included as part of the instant invention.

TABLE 1
Preferred Human DNA Codons

| Amino Acids | | | Codons |
|---|---|---|---|
| Alanine | Ala | A | GCC GCT GCA GCG |
| Cysteine | Cys | C | TGC TGT |
| Aspartic acid | Asp | D | GAC GAT |
| Glutamic acid | Glu | E | GAG GAA |
| Phenylalanine | Phe | F | TTC TTT |
| Glycine | Gly | G | GGC GGG GGA GGT |
| Histidine | His | H | CAC CAT |
| Isoleucine | Ile | I | ATC ATT ATA |
| Lysine | Lys | K | AAG AAA |
| Leucine | Leu | L | CTG CTC TTG CTT CTA TTA |
| Methionine | Met | M | ATG |
| Asparagine | Asn | N | AAC AAT |
| Proline | Pro | P | CCC CCT CCA CCG |
| Glutamine | Gln | Q | CAG CAA |
| Arginine | Arg | R | CGC AGG CGG AGA CGA CGT |
| Serine | Ser | S | AGC TCC TCT AGT TCA TCG |
| Threonine | Thr | T | ACC ACA ACT ACG |
| Valine | Val | V | GTG GTC GTT GTA |
| Tryptophan | Trp | W | TGG |
| Tyrosine | Tyr | Y | TAC TAT |

A number of additional embodiments in the context of nucleic acids are discussed below.

A. Vectors

RNA molecules, peptides and polypeptides may be encoded by a nucleic acid molecule comprised in a vector. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., (1989) and Ausubel et al., 1996, both incorporated herein by reference. A targeting molecule is one that directs the modified polypeptide to a particular organ, tissue, cell, or other location in a subject's body.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of RNA molecules used in methods of the invention. Expression vectors can contain a variety of “control sequences,” which refer to nucleic acid sequences necessary for the transcription and possibly translation of an operably linked coding sequence in a particular host organism. For instance, in some embodiments of the invention, there may sequences to allow for in vitro transcription of a sequence. In particular embodiments, the expression vector may contain an Sp6, T3, or T7 promoter. In addition to control sequences that govern transcription and translation, vectors and expression vectors may contain nucleic acid sequences that serve other functions as well and are described infra.

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” e.g., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202; U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it may be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

In certain embodiments of the invention, a vector may also include one or more of: an ATG initiation signal, internal ribosome binding sites, multiple cloning site (MCS), splicing site, termination signal, polyadenylation signal, origin of replication, or selectable or screenable marker (drug resistance marker, enzymatic marker, colorimetric marker, fluorescent marker).

In certain embodiments of the invention, the expression vector comprises a therapeutic gene for example, a vector may comprise the coding sequence for VEGF. This kind of vector may be useful in the treatment for example of myocardial infection. In some other cases a therapeutic expression vector may encode a HIF-1α gene. It is additionally contemplated that an expression vector for use in the current invention may encode an angiogenic factor, an anti-angiogenic factor, an interferon, a cytokine, a chemokine, a tumor suppressor, a protein kinase, a protein phosphotase, a cell surface receptor (or the extra cellular domain thereof), a growth factor or an enzyme.

B. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. Such a host cell would be considered recombinant if the heterologous nucleic acid sequence was the product of recombinant DNA technology. A host cell can, and has been, used as a recipient for vectors. A host cell may be “transfected” or “transformed,” which refers to a process by which exogenous nucleic acid, such as a modified protein-encoding sequence, is transferred or introduced into the host cell. A transformed cell includes the primary subject cell and its progeny.

Host cells may be derived from prokaryotes such as bacteria or eukaryotes, including yeast cells, insect cells, and mammalian cells, depending upon whether the desired result is replication of the vector or expression of part or all of the vector-encoded nucleic acid sequences. In certain embodiments, the cell is an embryonic stem cell, such as from a mouse.

Numerous cell lines and cultures are available for use as a host cell, and they can be obtained through the American Type Culture Collection (ATCC), which is an organization that serves as an archive for living cultures and genetic materials (World Wide Web at atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include but are not limited to XL-10-Gold and SURE 2 (Stratagene), which have been employed in the Examples. Additional bacterial cells are DH5α, JM109, and KC8, as well as a number of commercially available bacterial hosts such as SURE® Competent Cells and SOLOPACK™ Gold Cells (STRATAGENE®, La Jolla, Calif.). Alternatively, bacterial cells such as E. coli LE392 could be used as host cells for phage viruses. Appropriate yeast cells include Saccharomyces cerevisiae, Saccharomyces pombe, and Pichia pastoris.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

C. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. For example, high yield expression in sect cells such as SF-9 cells, may be accomplished by baculoviral expression systems. Another useful eukaryotic expression system is yeast which can be used to produce relatively large amounts of protein at a low cost. Many such systems are commercially and widely available.

D. Antisense Molecules, Ribozymes, and siRNA

In some embodiments of the invention, therapeutic nucleic acids are nucleic acid molecules with complementarity to target molecules. Such nucleic acids include antisense molecules, ribozymes, and siRNAs that are targeted to particular sequences based on the desired goal. In certain embodiments, for instance, a Notch may be inhibited or inactivated using an siRNA that targets a component of the Notch activation pathway, such as γ-secretase. Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNAs, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs may include regions complementary to intron/exon splice junctions. Thus, antisense constructs with complementarity to regions within 50-200 bases of an intron-exon splice junction may be used. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

The use of ribozymes is claimed in the present application. The following information is provided in order to compliment the earlier section and to assist those of skill in the art in this endeavor.

Ribozymes are RNA-protein complexes that cleave nucleic acids in a site-specific fashion. Ribozymes have specific catalytic domains that possess endonuclease activity (Kim and Cech, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence specific cleavage/ligation reactions involving nucleic acids (Joyce, 1989; Cech et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990; Sioud et al., 1992). Recently, it was reported that ribozymes elicited genetic changes in some cell lines to which they were applied; the altered genes included the oncogenes H-ras, c-fos and genes of HIV. Most of this work involved the modification of a target mRNA, based on a specific mutant codon that is cleaved by a specific ribozyme. In light of the information included herein and the knowledge of one of ordinary skill in the art, the preparation and use of additional ribozymes that are specifically targeted to a given gene will now be straightforward.

Several different ribozyme motifs have been described with RNA cleavage activity (reviewed in Symons, 1992). Examples that would be expected to function equivalently for the down regulation of AR include sequences from the Group I self splicing introns including tobacco ringspot virus (Prody et al., 1986), avocado sunblotch viroid (Palukaitis et al., 1979 and Symons, 1981), and Lucerne transient streak virus (Forster and Symons, 1987). Sequences from these and related viruses are referred to as hammerhead ribozymes based on a predicted folded secondary structure.

Other suitable ribozymes include sequences from RNase P with RNA cleavage activity (Yuan et al., 1992, Yuan and Altman, 1994), hairpin ribozyme structures (Berzal-Herranz et al., 1992; Chowrira et al., 1993) and hepatitis δ virus based ribozymes (Perrotta and Been, 1992). The general design and optimization of ribozyme directed RNA cleavage activity has been discussed in detail (Haseloff and Gerlach, 1988, Symons, 1992, Chowrira, et al., 1994, and Thompson, et al., 1995).

The other variable on ribozyme design is the selection of a cleavage site on a given target RNA. Ribozymes are targeted to a given sequence by virtue of annealing to a site by complimentary base pair interactions. Two stretches of homology are required for this targeting. These stretches of homologous sequences flank the catalytic ribozyme structure defined above. Each stretch of homologous sequence can vary in length from 7 to 15 nucleotides. The only requirement for defining the homologous sequences is that, on the target RNA, they are separated by a specific sequence which is the cleavage site. For hammerhead ribozymes, the cleavage site is a dinucleotide sequence on the target RNA, uracil (U) followed by either an adenine, cytosine or uracil (Perriman et al., 1992; Thompson et al., 1995). The frequency of this dinucleotide occurring in any given RNA is statistically 3 out of 16. Therefore, for a given target messenger RNA of 1000 bases, 187 dinucleotide cleavage sites are statistically possible.

Designing and testing ribozymes for efficient cleavage of a target RNA is a process well known to those skilled in the art. Examples of scientific methods for designing and testing ribozymes are described by Chowrira et al., (1994) and Lieber and Strauss (1995), each incorporated by reference. The identification of operative and preferred sequences for use in targeted ribozymes is simply a matter of preparing and testing a given sequence, and is a routinely practiced “screening” method known to those of skill in the art.

An RNA molecule capable of mediating RNA interference in a cell is referred to as “siRNA.” Elbashir et al. (2001) discovered a clever method to bypass the anti viral response and induce gene specific silencing in mammalian cells. Several 21-nucleotide dsRNAs with 2 nucleotide 3′ overhangs were transfected into mammalian cells without inducing the antiviral response. The small dsRNA molecules (also referred to as “siRNA”) were capable of inducing the specific suppression of target genes.

In the context of the present invention, siRNA directed against angiogenic factors, heat shock factors, and oncogene transcripts are specifically contemplated. For example, a siRNA may be directed against VEGF, heat shock protein 70 (HSP70), HSP90, ubiquitin or MMP. The siRNA can target a particular sequence because of a region of complementarity between the siRNA and the RNA transcript encoding the polypeptide whose expression will be decreased, inhibited, or eliminated.

An siRNA may be a double-stranded compound comprising two separate, but complementary strands of RNA or it may be a single RNA strand that has a region that self-hybridizes such that there is a double-stranded intramolecular region of 7 basepairs or longer (see Sui et al., 2002 and Brummelkamp et al., 2002 in which a single strand with a hairpin loop is used as a dsRNA for RNAi). In some cases, a double-stranded RNA molecule may be processed in the cell into different and separate siRNA molecules.

In some embodiments, the strand or strands of dsRNA are 100 bases (or basepairs) or less, in which case they may also be referred to as “siRNA.” In specific embodiments the strand or strands of the dsRNA are less than 70 bases in length. With respect to those embodiments, the dsRNA strand or strands may be from 5-70, 10-65, 20-60, 30-55, 40-50 bases or basepairs in length. A dsRNA that has a complementarity region equal to or less than 30 basepairs (such as a single stranded hairpin RNA in which the stem or complementary portion is less than or equal to 30 base pairs) or one in which the strands are 30 bases or fewer in length is specifically contemplated, as such molecules evade a mammalian's cell antiviral response. Thus, a hairpin dsRNA (one strand) may be 70 or fewer bases in length with a complementary region of 30 base pairs or fewer.

Methods of using siRNA to achieve gene silencing are discussed in WO 03/012052, which is specifically incorporated by reference herein. Designing and testing siRNA for efficient inhibition of expression of a target polypeptide is a process well known to those skilled in the art. Their use has become well known to those of skill in the art. The techniques described in U.S. Patent Publication No. 20030059944 and 20030105051 are incorporated herein by reference. Furthermore, a number of kits are commercially available for generating siRNA molecules to a particular target, which in this case includes AR, NF-κB, and TNF-α. Kits such as Silencer™ Express, Silencer™ siRNA Cocktail, Silencer™ siRNA Construction, MEGAScript® RNAi are readily available from Ambion, Inc.

E. Therapeutic Genes

In certain aspects of the invention, a therapeutic nucleic acid may comprise an RNA or DNA expression vector that can mediate expression of a therapeutic gene. The term “gene” is used for simplicity to refer to a functional protein, polypeptide, or peptide-encoding unit. “Therapeutic gene” is a gene which can be administered to a subject for the purpose of treating or preventing a disease. For example, a therapeutic gene can be a gene administered to a subject for treatment or prevention of cancer. Examples of therapeutic genes include, but are not limited to, Rb, CFTR, p16, p21, p27, p57, p73, C-CAM, APC, CTS-1, zac1, scFV ras, DCC, NF-1, NF-2, WT-1, MEN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, GM-CSF, G-CSF, thymidine kinase, mda7, fus, interferon α, interferon β, interferon γ, ADP, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, YES, MADH4, RB1, TP53, WT1, TNF, BDNF, CNTF, NGF, IGF, GMF, aFGF, bFGF, NT3, NTS, ApoAI, ApoAIV, ApoE, Rap1A, cytosine deaminase, Fab, ScFv, BRCA2, zac1, ATM, HIC-1, DPC-4, FHIT, PTEN, ING1, NOEY1, NOEY2, OVCA1, MADR2, 53BP2, IRF-1, Rb, zac1, DBCCR-1, rks-3, COX-1, TFPI, PGS, Dp, E2F, ras, myc, neu, raf, erb, fms, trk, ret, gsp, hst, abl, E1A, p300, VEGF, FGF, thrombospondin, BAI-1, GDAIF, or MCC.

Other examples of therapeutic genes include genes encoding enzymes. Examples include, but are not limited to, ACP desaturase, an ACP hydroxylase, an ADP-glucose pyrophorylase, an ATPase, an alcohol dehydrogenase, an amylase, an amyloglucosidase, a catalase, a cellulase, a cyclooxygenase, a decarboxylase, a dextrinase, an esterase, a DNA polymerase, an RNA polymerase, a hyaluron synthase, a galactosidase, a glucanase, a glucose oxidase, a GTPase, a helicase, a hemicellulase, a hyaluronidase, an integrase, an invertase, an isomerase, a kinase, a lactase, a lipase, a lipoxygenase, a lyase, a lysozyme, a pectinesterase, a peroxidase, a phosphatase, a phospholipase, a phosphorylase, a polygalacturonase, a proteinase, a peptidease, a pullanase, a recombinase, a reverse transcriptase, a topoisomerase, a xylanase, a reporter gene, an interleukin, or a cytokine.

Further examples of therapeutic genes include the gene encoding carbamoyl synthetase I, ornithine transcarbamylase, arginosuccinate synthetase, arginosuccinate lyase, arginase, fumarylacetoacetate hydrolase, phenylalanine hydroxylase, alpha-1 antitrypsin, glucose-6-phosphatase, low-density-lipoprotein receptor, porphobilinogen deaminase, factor VIII, factor IX, cystathione beta.-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, beta.-glucosidase, pyruvate carboxylase, hepatic phosphorylase, phosphorylase kinase, glycine decarboxylase, H-protein, T-protein, Menkes disease copper-transporting ATPase, Wilson's disease copper-transporting ATPase, cytosine deaminase, hypoxanthine-guanine phosphoribosyltransferase, galactose-1-phosphate uridyltransferase, phenylalanine hydroxylase, glucocerbrosidase, sphingomyelinase, α-L-iduronidase, glucose-6-phosphate dehydrogenase, HSV thymidine kinase, or human thymidine kinase.

Therapeutic genes also include genes encoding hormones. Examples include, but are not limited to, genes encoding growth hormone, prolactin, placental lactogen, luteinizing hormone, follicle-stimulating hormone, chorionic gonadotropin, thyroid-stimulating hormone, leptin, adrenocorticotropin, angiotensin I, angiotensin II, β-endorphin, β-melanocyte stimulating hormone, cholecystokinin, endothelin I, galanin, gastric inhibitory peptide, glucagon, insulin, lipotropins, neurophysins, somatostatin, calcitonin, calcitonin gene related peptide, β-calcitonin gene related peptide, hypercalcemia of malignancy factor, parathyroid hormone-related protein, parathyroid hormone-related protein, glucagon-like peptide, pancreastatin, pancreatic peptide, peptide YY, PHM, secretin, vasoactive intestinal peptide, oxytocin, vasopressin, vasotocin, enkephalinamide, metorphinamide, alpha melanocyte stimulating hormone, atrial natriuretic factor, amylin, amyloid P component, corticotropin releasing hormone, growth hormone releasing factor, luteinizing hormone-releasing hormone, neuropeptide Y, substance K, substance P, or thyrotropin releasing hormone.

### II. PROTEINACEOUS COMPOSITIONS

In certain embodiments, the present invention concerns compositions comprising at least one proteinaceous molecule, such as elastin-like polypeptides. As used herein, a “proteinaceous molecule,” “proteinaceous composition,” “proteinaceous compound,” “proteinaceous chain” or “proteinaceous material” generally refers, but is not limited to, a protein molecule containing at least one polypeptide with multiple amino acids. The protein may contain more than one polypeptide, such as a dimer or trimer or other tertiary structure. In some embodiments, a protein refers to a polypeptide that has 3 amino acids or more or to a peptide of from 3 to 100 amino acids. All the “proteinaceous” terms described above may be used interchangeably herein. In the case of a protein composed of a single polypeptide, the terms “polypeptide” and “protein” are used interchangeably.

In certain embodiments the size of the at least one proteinaceous molecule may comprise, or be at most or at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600 or greater amino molecule residues, and any range derivable therein. Moreover, it may contain such lengths of contiguous amino acids from a polypeptide provided herein, such as an elastin polymer.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino molecule sequences comprising at least one of the 20 common amino acids in naturally synthesized proteins, or at least one modified or unusual amino acid, including but not limited to those shown on Table 2 below.

TABLE 2
Modified and Unusual Amino Acids

| Abbr. | Amino Acid |
|---|---|
| Aad | 2 Aminoadipic acid |
| Baad | 3 Aminoadipic acid |
| Bala | β alanine, β Amino propionic acid |
| Abu | 2 Aminobutyric acid |
| 4Abu | 4 Aminobutyric acid, piperidinic acid |
| Acp | 6 Aminocaproic acid |
| Ahe | 2 Aminoheptanoic acid |
| Aib | 2 Aminoisobutyric acid |
| Baib | 3 Aminoisobutyric acid |
| Apm | 2 Aminopimelic acid |
| Dbu | 2,4 Diaminobutyric acid |
| Des | Desmosine |
| Dpm | 2,2′ Diaminopimelic acid |
| Dpr | 2,3 Diaminopropionic acid |
| EtGly | N Ethylglycine |
| EtAsn | N Ethylasparagine |
| Hyl | Hydroxylysine |
| AHyl | allo Hydroxylysine |
| 3Hyp | 3 Hydroxyproline |
| 4Hyp | 4 Hydroxyproline |
| Ide | Isodesmosine |
| AIle | allo Isoleucine |
| MeGly | N Methylglycine, sarcosine |
| MeIle | N Methylisoleucine |
| MeLys | 6 N Methyllysine |
| MeVal | N Methylvaline |
| Nva | Norvaline |
| Nle | Norleucine |
| Orn | Ornithine |

In certain embodiments the proteinaceous composition comprises at least one protein, polypeptide or peptide. In further embodiments the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's Genbank and GenPept databases (on the World Wide Web at ncbi.nlm.nih.gov/). The coding regions for these known genes may be amplified and/or expressed using the techniques disclosed herein or as would be know to those of ordinary skill in the art. Alternatively, various commercial preparations of proteins, polypeptides and peptides are known to those of skill in the art.

In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

In certain embodiments, the proteinaceous composition may comprise at least one antibody, for example, an antibody against a tumor antigen, which may be used to determine whether it is sequestered. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, single domain antibodies (DABs), Fv, scFv (single chain Fv), and the like. The techniques for preparing and using various antibody based constructs and fragments are well known in the art. Means for preparing and characterizing antibodies are also well known in the art (see, e.g., Harlow et al., 1988; incorporated herein by reference).

It is contemplated that virtually any protein, polypeptide or peptide containing component may be used in the compositions and methods disclosed herein. However, it is preferred that the proteinaceous material is biocompatible. In certain embodiments, it is envisioned that the formation of a more viscous composition will be advantageous in that will allow the composition to be more precisely or easily applied to the tissue and to be maintained in contact with the tissue throughout the procedure. In such cases, the use of a peptide composition, or more preferably, a polypeptide or protein composition, is contemplated. Ranges of viscosity include, but are not limited to, about 40 to about 100 poise. In certain aspects, a viscosity of about 80 to about 100 poise is preferred.

In some further aspects of the invention, it will be understood that the sequence of an ELP domain may be modified, for example, to change the phase transition characteristics of an ELP, ELP composition or bioplex. For instance, in some cases, an ELP domain comprises the sequence VPGXG, wherein X is any amino acid except proline. By substituting of different amino acids at the X position the characteristics of an ELP domain may be modified. For example, in the case where a lower transition temperature is desired more hydrophobic residues may be substituted at X. Conversely, to increase the transition temperature less hydrophobic residues may be substituted at the X position. The importance of hydrophobicity or the hydropathic amino acid index in conferring biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (0.5); histidine −0.5); cysteine (−1.0); methionine (−1.3); valine (−1.5); leucine (−1.8); isoleucine (−1.8); tyrosine (2.3); phenylalanine (−2.5); tryptophan (−3.4). Thus, it will be understood that when the amino acid at position X has a high hydrophilicity value ELP transition temperature can be raised whereas to lower the transition temperature amino acids with lower hydrophilicity values may be used.

It will also be understood that the transition temperature of an ELP domain, ELP composition or bioplex may be modified by changing the number of elastin-like repeats in an ELP domain. For example, in order to raise the transition temperature conferred by an ELP domain the number of ELP repeats may be reduced. Conversely, increasing the number of ELP repeats in an ELP domain will generally decrease the transition temperature of an ELP domain, ELP composition or bioplex.

In additional aspects of the invention polypeptides domains may be further modified by amino substitutions, for example by substituting an amino acid at one or more positions with an amino acid having a similar hydrophilicity (see above). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Thus such conservative substitution can be made in ELP domain, cell targeting domain, a membrane translocation domain, a therapeutic polypeptide domain or a nucleic acid binding domain and such substitutions will likely only have minor effects on their activity. For instance, substitution of amino acids whose hydrophilicity values are within ±2 are preferred, those that are within ±1 are particularly preferred, and those within ±0.5 are even more particularly preferred. Thus, any of the polypeptide domains described herein may be modified by the substitution of an amino acid, for different, but homologous amino acid with a similar hydrophilicity value. Amino acids with hydrophilicities within +/−1.0, or +/−0.5 points are considered homologous.

In certain embodiments, a peptide or polypeptide may contain an amino acid sequence that is identical or similar to a reference sequence or a particular region of the reference sequence. In certain embodiments a peptide or polypeptide has at least or most 60, 65, 70, 75, 80, 85, 90, 95, 96, 97, 98, 99, 99.5, 100% identity with respect to the amino acid sequence of a particular polypeptide or within a region of the particular polypeptide. In some cases, an ELP domain sequence may modified to more closely match the sequence of a human elastin domain, comprising a repeats of the sequence VPGVG. Such modifications may be made, for example, to further reduce the immunogenicity of an ELP domain, or ELP composition (e.g., ELP fusion proteins). For instance, in some embodiments of the invention, there an ELP domain may be defined a at least about 60, 65, 70, 75, 80, 85, 90 or 95% identical to the human elastin repeat sequence.

In the case of similar amino acids, certain amino acids can be substituted for one another with minimal effect on protein function. Amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include those in the table below.

TABLE 3
Example amino acid substitutions

| Original Residue | Exemplary Substitutions |
|---|---|
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Met; Leu; Tyr |
| Ser | Thr |
| Thr | Ser |
| Trp | Tyr |
| Tyr | Trp; Phe |
| Val | Ile; Leu |

Accordingly, sequences that have between about 70% and about 80%, between about 81% and about 90%; or between about 91% and about 99%; of amino acids that are identical or functionally equivalent to the amino acids of a reference polypeptide sequence are included as part of the invention.

Another embodiment for the preparation of polypeptides according to the invention is the use of peptide mimetics. Mimetics are peptide-containing molecules that mimic elements of protein secondary structure. See, e.g., Johnson (1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of the original protein, but with altered and even improved characteristics.

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes such as a hydrolase, glycosylation domains, cellular targeting signals or transmembrane regions.

In certain aspects of the invention, the charge of an amino acid in a polypeptide is an important characteristic. For example, in the case on an ELP domain charged amino acids can alter the transition temperature conferred by the domain. Another example is a nucleic acid binding domain. In certain aspects, cationic amino acids are use to bind nucleic acids via electrostatic interactions. In these cases, the pH of a solution is important in that it can alter the charge of amino acid and this the characteristics of ELP compositions of the invention (e.g., nucleic acid interaction may be destabilized or ELP transition temperature altered). Table 4 below summarizes the pKa values for the common 20 amino acids an can be used to determine the percentage of any particular amino acid that will charged at a give pH. Of course, it will be understood that in many cases a neutral or physiological pH will be preferred.

TABLE 4
Amino Acid pKa values

| | Carboxylic | | |
|---|---|---|---|
| A.A. | acid | Amine | Side Chain |
| A | 2.3 | 9.9 | — |
| C | 1.8 | 10.8 | 8.6 |
| D | 2.0 | 10.0 | 4.5 |
| E | 2.2 | 9.7 | 4.5 |
| F | 1.8 | 9.1 | — |
| G | 2.4 | 9.8 | — |
| H | 1.8 | 9.2 | 6.8 |
| I | 2.4 | 9.7 | — |
| K | 2.2 | 9.2 | 10.1 |
| L | 2.4 | 9.6 | — |
| M | 2.3 | 9.2 | — |
| N | 2.0 | 8.8 | — |
| P | 2.0 | 10.6 | — |
| Q | 2.2 | 9.1 | — |
| R | 1.8 | 9.0 | 12.5 |
| S | 2.1 | 9.2 | — |
| T | 2.6 | 10.4 | — |
| V | 2.3 | 9.6 | — |
| W | 2.4 | 9.4 | — |
| Y | 2.2 | 9.1 | 9.8 |

A. Protein Purification

In some embodiments, it may be desirable to purify a protein, for example, an ELP composition fusion protein. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide or polypeptide are filtration, ion-exchange chromatography, exclusion chromatography, polyacrylamide gel electrophoresis, affinity chromatography, or isoelectric focusing. A particularly efficient method of purifying peptides is fast protein liquid chromatography or even HPLC. In the case of ELP compositions protein purification may also be aided by the thermal transition properties of the ELP domain as described in U.S. Pat. No. 6,852,834.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

B. Antibodies

Another embodiment of the present invention may involve antibodies. In some cases, for example an antibody may be used a cell targeting domain in a ELP composition of the invention. Such antibodies may be made against virtually any antigen of interest according to methods that are well known to those in the art.

mAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified polypeptide, peptide or domain, be it a wild-type or mutant composition. The immunizing composition is administered in a manner effective to stimulate antibody producing cells.

Antibodies may be further purified, if desired, using filtration, centrifugation and various chromatographic methods such as HPLC or affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, antibody fragments encompassed by the present invention can be synthesized using an automated peptide synthesizer.

It also is contemplated that a molecular cloning approach may be used to generate antibodies.

“Humanized” antibodies are also contemplated, as are chimeric antibodies from mouse, rat, or other species, bearing human constant and/or variable region domains, bispecific antibodies, recombinant and engineered antibodies and fragments thereof. The techniques for producing humanized immunoglobulins are well known to those of skill in the art. For example U.S. Pat. No. 5,693,762 discloses methods for producing, and compositions of, humanized immunoglobulins having one or more complementarity determining regions (CDR's). When combined into an intact antibody, the humanized immunoglobulins are substantially non immunogenic in humans and retain substantially the same affinity as the donor immunoglobulin to the antigen, such as a protein or other compound containing an epitope. Examples of other teachings in this area include U.S. Pat. Nos. 6,054,297; 5,861,155; and 6,020,192, all specifically incorporated by reference. Methods for the development of antibodies that are “custom-tailored” to the patient's disease are likewise known and such custom-tailored antibodies are also contemplated.

### III. THERAPEUTIC AND PREVENTATIVE METHODS

A. Pharmaceutical Formulations, Delivery, and Treatment Regimens

In certain embodiments of the invention, there are methods of achieving a therapeutic effect, such as treatment of a disease by ELP delivery of therapeutic compositions such as nucleic acids.

An effective amount of the pharmaceutical composition, generally, is defined as that amount sufficient to detectably and repeatedly to ameliorate, reduce, minimize or limit the extent of the disease or its symptoms. More rigorous definitions may apply, including elimination, eradication or cure of disease.

Administration and Dosage

To effect a physiological or therapeutic effect using the methods and compositions of the present invention, one would generally contact a cell with the therapeutic compound or candidate therapeutic agent, such as a protein or an expression construct encoding a protein. The routes of administration will vary, naturally, with the location and nature of the lesion, and include, e.g., intradermal, transdermal, parenteral, intravenous, intramuscular, intranasal, subcutaneous, percutaneous, intratracheal, intraperitoneal, intratumoral, perfusion, lavage, via inhalation (e.g., as an aerosol), direct injection, and oral administration and formulation.

For example, in the case where a drug delivery vehicle of the invention is administered as an aerosol the unique thermal transition properties conferred by the ELP domain may offer certain therapeutic advantages. For example, a delivery vehicle may be dispersed in a liquid prior to administration (e.g., at a temperature below the transition temperature for the delivery vehicle). Then for administration temperature may be raised during or prior to aerosolization, thereby enabling methods for adjusting the size of aerosol particulates based on the magnitude of the temperature increase. Thus, methods for delivery of therapeutics (e.g., GA) via inhaled drug delivery vehicles may be far more efficient than prior methods that do not allow for the modulation of the delivery particle size.

To effect a therapeutic benefit with respect to a genetic disease, condition, or disorder, one would contact a cell having the relevant nucleic acids and exhibiting the unwanted phenotype with the therapeutic compound. Any of the formulations and routes of administration discussed with respect to the treatment or diagnosis of cancer may also be employed with respect to such diseases and conditions.

Continuous administration also may be applied where appropriate. Delivery via syringe or catherization is preferred. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs. It is further contemplated that limb perfusion may be used to administer therapeutic compositions of the present invention.

Treatment regimens may vary as well, and often depend on disease progression, and health and age of the patient. Obviously, certain conditions, disorders, or diseases will require more aggressive treatment, while at the same time, certain patients cannot tolerate more taxing protocols. The clinician will be best suited to make such decisions based on the known efficacy and toxicity (if any) of the therapeutic formulations.

The treatments may include various “unit doses.” Unit dose is defined as containing a predetermined quantity of the therapeutic composition. The quantity to be administered, and the particular route and formulation, are within the skill of those in the clinical arts. A unit dose need not be administered as a single injection but may comprise continuous infusion over a set period of time. Unit dose of the present invention may conveniently be described in terms of, for example, mg or μg amounts of ELP composition.

It is specifically contemplated that the candidate substance, candidate therapeutic agent, or ELP is administered to the subject over a period of time of about, of at least about, or at most about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96 hours or more, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 days or more, 1, 2, 3, 4, 5, 6, 7, 8, 9, 10, 11, 12 weeks or more, 1, 2, 3, 4 months or more, or any range derivable therein.

In other embodiments, methods involve administering a dose or dosage of a compound or agent to the subject. It will be understood that the amount given to the subject may be dependent on the weight of the subject and this may be reflected in the amount given in a day (e.g., a 24-hour period). In some embodiments, a subject is given about, less than about, or at most about 0.005, 0.05, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 100, 110, 120, 130, 140, 150 nM/kg/day, or any range derivable therein. Alternatively, the amount of compound or agent that is administered can be expressed in terms of nanogram (ng). In certain embodiments, the amount given is about, less than about, or at most about 0.005, 0.05, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 ng/kg/day, or any range derivable therein.

B. Injectable Compositions and Formulations

Pharmaceutical compositions disclosed herein may alternatively be administered parenterally, intravenously, intradermally, intramuscularly, transdermally or even intraperitoneally as described in U.S. Pat. No. 5,543,158; U.S. Pat. No. 5,641,515 and U.S. Pat. No. 5,399,363 (each specifically incorporated herein by reference in its entirety).

Injection of nucleic acid, small molecules, or proteins may be delivered by syringe or any other method used for injection of a solution, as long as the expression construct can pass through the particular gauge of needle required for injection. A novel needleless injection system has recently been described (U.S. Pat. No. 5,846,233) having a nozzle defining an ampule chamber for holding the solution and an energy device for pushing the solution out of the nozzle to the site of delivery. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

In certain embodiments, the agent or substance may be administered to the subject in prodrug form, meaning that it will become the active agent or substance once it has entered the subject's body, or a certain body cavity or cell.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

In certain embodiments, the present invention concerns compositions comprising one or more lipids associated with a nucleic acid, an amino acid molecule, such as a peptide, or another small molecule compound. A lipid is a substance that is characteristically insoluble in water and extractable with an organic solvent. Compounds than those specifically described herein are understood by one of skill in the art as lipids, and are encompassed by the compositions and methods of the present invention. A lipid component and a non-lipid may be attached to one another, either covalently or non-covalently.

It is contemplated that a liposome composition may comprise additional materials for delivery to a tissue. For example, in certain embodiments of the invention, the lipid or liposome may be associated with a hemagglutinating virus (HVJ). This has been shown to facilitate fusion with the cell membrane and promote cell entry of liposome-encapsulated DNA (Kaneda et al., 1989). In another example, the lipid or liposome may be complexed or employed in conjunction with nuclear non-histone chromosomal proteins (HMG-1) (Kato et al., 1991). In yet further embodiments, the lipid may be complexed or employed in conjunction with both HVJ and HMG-1.

C. Hyperthermia Therapy

The in vitro and in vivo uses of hyperthermia include several effects (both beneficial and detrimental) that should be addressed prior to application of therapy. Hyperthermia has long been used for the treatment of cancer; moreover, several studies show promising synergistic effects of hyperthermia combined with chemo- and/or radiotherapy. For example, 43° C. hyperthermia has been shown to increase the thermal enhancement ratio (TER, ratio of cell viability with hyperthermia to co-administered hyperthermia and drug) of Cisplatin by ˜1.4-5.0 fold in mouse mammary tumors (Beketic-Oreskovic et al., 1997). While it is apparent that hyperthermia can induce cancer cell death at T>43° C., Hsp activation has been shown to convert some cells to a hyperthermia-insensitive or “thermotolerant” state, effectively ending the hyperthermic apoptotic pathway (Hildebrandt et al., 2002).

Inducing hyperthermia in accordance with the methods of the present invention can be accomplished in a variety of manners. Essentially any technique that produces an appropriate increase in temperature in the tissue of interest can be used. Preferably, techniques of raising temperature in tissue that allow for maintaining the elevated temperature over a period of time are used.

Several methods of inducing hyperthermia in tissue have been described. U.S. Pat. No. 6,167,313 provides an overview of several techniques and methods. Any standard technique can be used to accomplish the desired hyperthermia. For example, an ultrasonic transducer can be employed to deliver a localized increase in tissue temperature. For an example of methods and apparatuses in accordance with this category, see U.S. Pat. No. 5,620,479. Alternatively, a technique commonly referred to as interstitial hyperthermia can be employed. Other alternative methods of inducing hyperthermia include exposing the tissue to microwave radiation (for example, see U.S. Pat. No. 5,861,021 and U.S. Pat. No. 5,922,013) or magnetic induction (see U.S. Pat. No. 6,167,313).

The method employed to induce hyperthermia can be optimized based upon the nature of the tissue of interest. For example, for deep tissues, such as a tumor in prostate tissue, interstitial hyperthermia will likely offer a better ability to control the hyperthermia. For surface tissues, a simple device, such as an ultrasonic transducer, will likely by sufficient.

D. Additional Combination Treatments

Administration of the therapeutic agent or substance of the present invention to a patient will follow general protocols for the administration of that particular secondary therapy, taking into account the toxicity, if any, of the treatment. It is expected that the treatment cycles would be repeated as necessary. It also is contemplated that various standard therapies, as well as surgical intervention, may be applied in combination with the described therapy.

### IV. EXAMPLES

The following examples are included to further illustrate various aspects of the invention. It should be appreciated by those of skill in the art that the techniques disclosed in the examples that follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Preparation of Geldanamycin (Ga) Derivatives

GA was obtained by fermentation of Streptomyces hygroscopicus subsp. geldanus (ATCC 55256) as described previously (DeBoer et al., 1970). Briefly, an ISP2 plate was streaked with a frozen spore suspension (1.5×10⁻⁶ mL⁻¹) and grown until distinct white colonies appeared (˜30° C., ca. 10 days). A single colony was used to inoculate 100 mL of fermentation medium (28° C., in dark, 270 rpm orbital shaker), and after 5 days GA production was verified by HPLC analysis of a 1:1 EtOAc broth extraction. A 2-L baffled Erlenmeyer flask with 25 g of glass beads and 0.5 L of fermentation medium was inoculated with 5 mL of inoculum (28° C., in dark, 270 rpm orbital shaker). The 5-day fermentation broth was centrifuged (10 min, 8000×g), filtered, and the filtrate extracted (1:1 EtOAc, 3×). Solids were lyophilized and extracted with MeOH. The crude organic extracts were reduced in vacuo, filtered, and purified over silica (1:1 EtOAc:hexanes, 2×). Final purification was done by reverse phase HPLC (Ace 5-μm C18 10×150 mm, MACMOD Analytical, Chadds Ford, Pa.) using a MeOH—5% HOAc gradient (70:30-99:1, 30° C., 305-nm detection). Purity: (HPLC) (Ace 3-μm C18 5×50 mm, 75-100% MeOH—50 mM pH 5.0 acetic acid, 50° C., 305-nm detection). Yield: 150-200 mg/L broth.

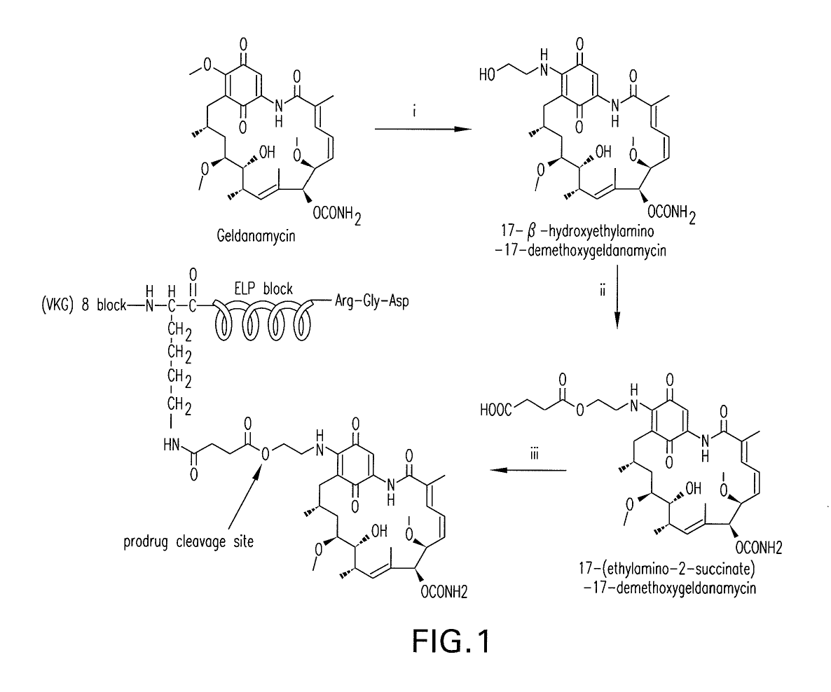

17-β-hydroxyethylamino-17-demethoxygeldanamycin (17-HEA-DMGA) was synthesized as described in U.S. Pat. No. 4,261,989 with slight modifications (FIG. 1, step i). Briefly, 170 mg of 1 (0.3 mmol) was dissolved in 15 mL CHCl₃ and 20 eq. 2-aminoethanol was added in one portion. The reaction was stirred in low light at RT until complete by TLC (ca. 2 hr) (95:5 CHCl₃:MeOH, Rf 0.17), extracted with 1 M HCl twice, dried over anhydrous NaSO₄, and evaporated to dryness. Yield: 170 mg, 95%.

17-(ethylamino-2-succinate)-17-demethoxygeldanamycin (17-EAS-DMGA) was prepared by reacting 17-HEA-DMGA with 2-hydroxyethylamine (FIG. 1, step ii). Briefly, 24 mg (0.04 mmol) of 2 was dissolved in 20 mL CHCl₃ and 0.2 eq. of N,N′-dimethylamino pyridine (DMAP) and 2 eq. 2-hydroxyethylamine were added to the 17-HEA2 DMGA solution. The reaction was stirred in low light at RT until complete by TLC (ca. 2 hr) (90:10 CHCl₃:MeOH, Rf 0.3), extracted with CHCl₃, dried over anhydrous NaSO₄, and evaporated to dryness. Yield: 45 mg, 95%.

### Example 2

### Preparation of Geldanamycin (Ga) Conjugates

GA-conjugated (VKG)₈ELP1-120 was synthesized by the coupling of 3 to (VKG)₈ELP1-120 as shown in FIG. 1, step iii. Briefly, 11 mg (0.02 mmol) of 3 was dissolved in 15 mL of DMSO, activated by adding 25.2 mg (2.2 mmol) of dicyclohexyl carbodiimide (DCC) and 23 mg (0.2 mmol) of N-hydroxy sulfosuccinimide at room temperature for 3 hr. The 50 mg (1 μM of NH2-group on lysine residue) of (VKG)₈ELP1-120 dissolved in DMSO (final content of water in (VKG)₈ELP1-120 solution was about 10%) and added to the activated GA solution. The reaction was carried out in the presence of 0.2 eq. DMAP (290 mg, 3.68 mmol) at room temperature for 5 hr followed by filtration through a 0.45 μm filter unit (Millipore, Bedford, Mass.) to remove insoluble materials. The conjugate was precipitated by adding 100 mL of acetone to the reaction mixture and isolated by centrifugation at 15000×g for 10 min. The GA-(VKG)₈ELP1-120 conjugate was dissolved in 1 mL PBS and stored at −80° C. Yield: 25 mg, 50% based upon the (VKG)₈ELP1-120 used.

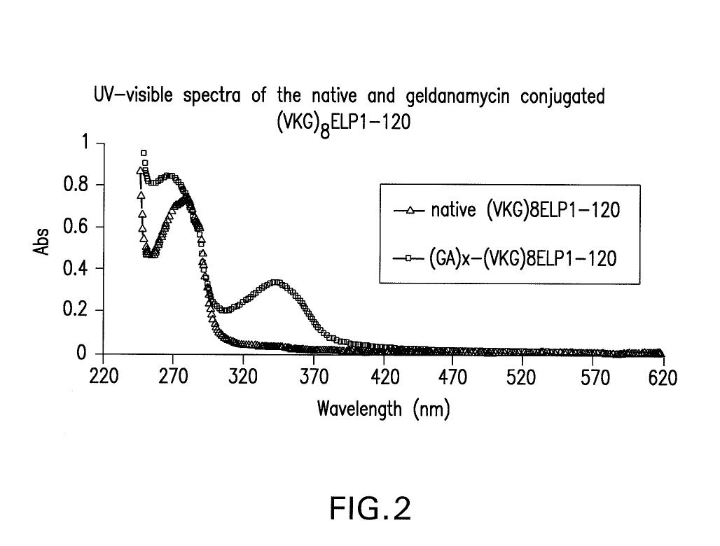

The UV-visible spectrum of GA-(VKG)₈ELP1-120 was characterized by UV-visible spectrophotometry to determine the GA conjugation ratio. As shown in FIG. 2, there is an increase in absorbance at 346 nm upon conjugation of GA to the lysine group of (VKG)₈ELP1-120. Using the ε₃₃₂=2.2×10⁴ M⁻¹cm⁻¹ of 17-(3-aminopropylamino)-17-demethoxygeldanamycin, the conjugation ratio was determined to be 0.93.

### Example 3

### Thermal Transition of GA-(VKG)₈ELP1-120

Free ELP has a sharp transition that occurs over a 1-2° C. range with a Tt value of 43.6° C. at 25 μM concentration. In contrast, this particular GA-ELP conjugate (GA-(VKG)₈ELP1-120) shows a broad thermal transition that occurs over a 10-12° C. range (FIG. 3). The Tt value of the GA-ELP conjugate decreased to 39.2° C.

### Example 4

### Cytotoxicity of GA-(VKG)₈ELP1-120

Test Compounds

A. 17-β-hydroxyethylamino-17-demethoxygeldanamycin (17-OHGA)

B. 17-GAOH-(VKG)8ELP1-120: conjugation ratio was 0.93 as determined by UV-visiblespectroscopy using the ε₃₃₂=2.2×10⁴ M⁻¹cm⁻¹ of 17-(3-aminopropylamino)-17-demethoxygeldanamycin.

Concentration of Stock Solutions

A. 17-GAOH: 1 mM, 0.1 mM, 0.01 mM, 1 μM, 0.1 μM, and 0.01 μM in DMSO

B. 17-GAOH-(VKG)₈ELP1-120: 0.1 mM, 0.01 mM, 1 μM, 0.1 μM, and 0.01 μM in PBS at pH 7.25

Cytotoxicity Test

MCF-7 human breast cancer cells (ATCC HTB-22) were plated in 96-well plates at an initial density of 5000 cells per well in 90 μL of RMPI 1640 supplemented with 10% fetal bovine serum, 100 IU penicillin, and 100 μg/mL streptomycin, 2 mM L-glutamine, and maintained at 37° C. and 5% CO₂ atmosphere. After 24 hr, stock solutions of 17-GAOH in DMSO and GA-(VKG)8ELP1-120 were diluted 10-fold with growth media and added to wells (6 wells) as 10-A aliquots (1% v/v final DMSO concentration). The cells were incubated with the compounds for 74 hr and the metabolic rate was determined using an XTT™ assay kit (Sigma) according to the manufacturer's instructions. The concentrations inhibiting cell growth by 50% (IC₅₀) were determined by fixed Hill slope regression with SIGMA PLOT™ 2004 (Systat Software, Inc.).

TABLE 5
Cytotoxicity of GA-(VKG)₈ELP1-120 in test 1.

| Final | 17-HEA-DMGA | | | GA-(VKG)₈ELP1-120 | | |
|---|---|---|---|---|---|---|
| Concentration | | | IC₅₀ | | | IC₅₀ |
| (nM) | Average | St. Dev. | (nM) | Average | St. Dev. | (nM) |
| 0 | 1684 | 52 | 855 | 1682 | 52 | 350 |
| 0.1 | 1158 | 115 | | 1605 | 59 | |
| 1 | 1288 | 147 | | 1679 | 47 | |
| 10 | 1274 | 113 | | 1616 | 77 | |
| 100 | 1588 | 136 | | 1592 | 24 | |
| 1,000 | 344 | 132 | | 116 | 58 | |

| | | | |
|---|---|---|---|
| 10,000 | 114 | 21 | Not tested |

TABLE 6
Cytotoxicity of GA-(VKG)₈ELP1-120 in test 2.

| Final | 17-HEA-DMGA | | | GA-(VKG)₈ELP1-120 | | |
|---|---|---|---|---|---|---|
| Concentration | | | IC₅₀ | | | IC₅₀ |
| (nM) | Average | St. Dev. | (nM) | Average | St. Dev. | (nM) |
| 0 | 603.0 | 63.8 | 731 | 1043.8 | 81.3 | 1042 |
| 50 | 788.5 | 240.7 | | 1373.1 | 51.8 | |
| 100 | 999.7 | 80.2 | | 1339.4 | 55.2 | |
| 250 | 774.3 | 146.4 | | 1252.7 | 84.5 | |
| 500 | 515.0 | 80.6 | | 1339.9 | 118.7 | |
| 1,000 | 297.5 | 90.3 | | 1059.6 | 39.4 | |
| 2,000 | 46.9 | 60.1 | | 436.8 | 44.3 | |

### Example 5

### Materials and Methods for Example 6

Materials and cell lines: Top10 cells and E. coli BLR(DE3) strain were purchased from Invitrogen (Carlsbad, Calif.) and Novagen (Madison, Wis.), respectively. T4 DNA ligase, restriction enzymes and pUC19 cloning vectors were from New England Biolabs (Beverly, Mass.). Calf intestinal alkaline phosphatase (CIP), pGL3-control plasmids, and the CellTiter-Glo kit were from Promega (Madison, Wis.). The pET-25b(+)SV2 and pUC19-ELP(1-30) plasmids were gifted by Prof. Ashutosh Chilkoti (Duke University). CircleGrow culture medium was from Q-BIOgene (Carlsbad, Calif.). Oligonucleotides were synthesized at the UW-Madison Biotechnology Center. Human breast cancer MCF-7 cells were from American Type Culture Collection (ATCC). Dulbecco's modified eagle medium (DMEM) and fetal bovine serum (FBS) were from Mediatech, Inc. (Herndon, Va.). Aminoacetaldehyde diethyl acetal, 2-aminoethanol, resazurin sodium salt, chloroform, and dimethyl sulfoxide were from Sigma-Aldrich (Milwaukee, Wis.). These chemicals were used without further purification. Biosynthesis of K8-ELP(1-60) block copolymers.

K8-ELP(1-60) block copolymers were synthesized in principle according to previous studies with recursive directional ligation albeit with slight modification. Briefly, oligonucleotides with forward and reverse DNA sequences of 5′-GTGGGTAAAAAAAAGAAAAAAAAAAAGAAAGGC-3′ and 5′-TTTCTTTTTTTTTTTCTTTTTTTTACCCACGCC-3′, respectively, were annealed to form a double-stranded DNA cassette with PflM I and Bgl I compatible ends. pUC19-ELP(1-30) was digested with PflM I and dephosphorylated using CIP. The linearized pUC19-ELP(1-30) vector was separated and purified by low melting agarose gel electrophoresis and a QIAGEN QIAquick gel extraction kit. The DNA cassette and the linearized pUC19-ELP(1-30) vector were ligated, followed by transformation into Top10 cells by heat shock. The cells were then spread on CircleGrow medium agar plates supplemented with ampicillin (100 μg/mL). After overnight incubation at 37° C., colonies were collected and incubated further for an additional 12 h. Plasmids were isolated using the QIAGEN Miniprep kit and K8-ELP(1-30) was excided with PflM I and Bgl I. The gel purified insert was again ligated into the linearized pUC19-ELP(1-30) vector, and clones were screened as previously described. The pET-25b(+)SV2 expression vector was modified with the forward and reverse DNA sequences of 5′-TATGAGCGGGCCGGGCTGGCCGTGATA-3′ and 5′-AGCTTATCACGGCCAGCCCGGCCCGCTCA-3′, respectively [pET-25b(+)HCl]. Oligonucleotides were annealed to form a double-stranded DNA cassette with Nde I and HinD III compatible ends and this cassette was inserted into the Nde I and HinD III restriction sites by restriction enzyme digestion and ligation. pET-25b(+)HCl was linearized by digestion with Sfi I and enzymatical dephosphorylation using CIP, followed by ligation with K8-ELP(1-60). The plasmid was transformed into the E. coli BLR (DE3) strain by heat shock and the K8-ELP(1-60) diblock copolymers were expressed at 37° C. for 24 h. The polymers were collected from E. coli lysate by the inverse transition cycling (ITC) method previously described [28]. The purity and molecular weight of the polymers were confirmed by SDS-PAGE and MALDI-TOF mass spectrometry. K8-ELP(1-60) block copolymers were dialyzed against distilled water, freeze-dried, and stored at −80° C. for future use.

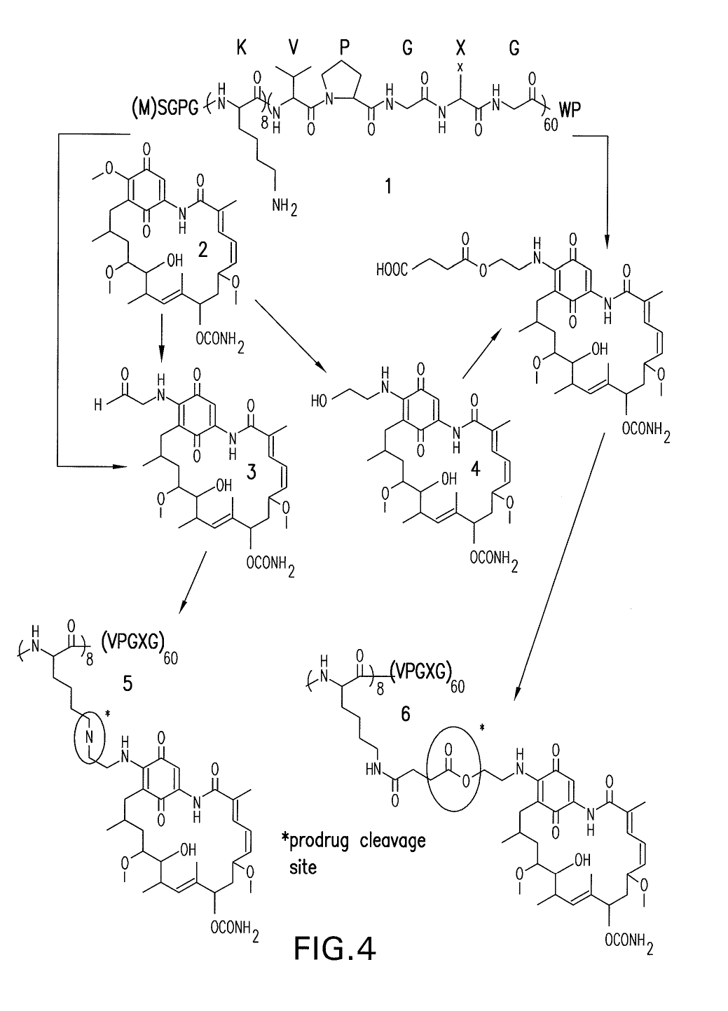

Synthesis of Geldanamycin (GA) derivatives: GA was obtained by fermentation of Streptomyces hygroscopicus subsp. geldanus. As shown in FIG. 4, the 17-methoxy position of 2 was modified with aldehyde and aminoethanol to prepare 3 and 4, respectively. 113 mg (0.2 mmol) of 1 was dissolved in 10 mL CHCl₃ after which 2-fold aminoacetaldehyde diethyl acetal or 2-aminoethanol was added to the solution. The reaction was allowed to proceed with stirring at 25° C. for 3 h. Purple products were purified and extracted by phase separation. The acetal groups of the intermediates of 3 were deprotected with 1 M HCl and the final product 3 was again extracted by phase separation. After solvent evaporation, GA derivatives were collected by freeze-drying. Purity of the products was confirmed by TLC (95:5 CHCl3:MeOH) with a UV light (254 nm). Preparation of [K8-ELP(1-60)]-GA conjugates [K8-ELP(1-60)]-GA conjugates 5 and 6 were prepared by conjugating 3 and 4 to the lysine groups of 1. In order to prepare 5, 50 mg of 1 and 2-fold of 3 with respect to the molar ratio of lysine groups of 1 were mixed in DMSO. The reaction was allowed to proceed at 25° C. for 3 days. 5 was collected by ether precipitation and freeze-drying. In the case of 6, 4 was activated further prior to conjugation. 30 mg (0.05 mmol) of 4 was dissolved in 25 mL CHCl₃. Dimethylamino pyridine and succinic anhydride were added to the solution with 0.2 and 2 equivalent molar amounts, respectively. Activated 4 was collected by phase separation and freeze-dried. 11 mg (0.02 mmol) of 4 was dissolved in 15 mL of DMSO, followed by adding 1.15 mg (0.1 mmol) of dicyclohexyl carbodiimide and 5.8 mg (0.05 mmol) of N-hydroxy sulfosuccinimide. The reaction was carried out at 25° C. for 3 h. 50 mg of 1 and 39 mg of dimethylamino pyridine were then added to this solution, followed by an additional 5 h reaction. After the reaction, insoluble impurities were filtered off and 6 was collected by precipitation in ether and freeze-drying.

UV-visible characterization and ¹H-NMR measurements: In order to determine the drug conjugation ratios of [K8-ELP(1-60)]-GA conjugates, UV-visible measurements were carried out using a Cary 100 UV-visible spectrophotometer (Walnut Creek, Calif.). [K8-ELP(1-60)]-GA conjugates were dissolved in PBS with various concentrations for the measurements. The drug conjugation ratio of [K8-ELP(1-60)]-GA conjugates was calculated from a calibration line of free GA derivatives with absorbance at 337 nm. Thermosensitive phase transition profiles were observed by monitoring optical density of the sample solutions at 350 nm while increasing the temperature from 25 to 90° C. (1° C./min). The transition temperature (Tt) was determined as the temperature that exhibits 50% of the maximum optical density. Samples were dissolved in d6-DMSO for ¹H-NMR analysis and data were collected at 25° C.

Dynamic light scattering (DLS) measurements: [K8-ELP(1-60)]-GA(CHO) and [K8-ELP(1-60)]-GA(OH) were dissolved in 2 mL PBS at 25 μM based on the polymer concentration, respectively. DLS measurements were carried out using a NICOMP 380 ZLS instrument, Particle Sizing Systems (Santa Barbara, Calif.). Particle sizes were measured at 25° C. and 80° C., while temperature was adjusted by a water bath. These temperatures were determined by considering the condition where phase transition remains suppressed and reaches plateau at 25 μM polymer concentrations. Data were acquired and processed by accompanying software ZPW388.

In vitro assay for hyperthermic combination chemotherapy: Human breast cancer MCF-7 cells were seeded on 96-well plates at a density of 3,000 cells/well. Cells were cultured at 37° C. and a 5% CO₂ atmosphere. After 24 h preincubation, the cell-culturing medium was replaced with 90 μL of fresh DMEM supplemented with 10% fetal bovine serum. 10 μL of samples with various concentrations were added to each well. Two sets of plates were prepared identically and one set of plates was incubated in a hot block chamber at 43° C. for 30 min for hyperthermic combination treatments. The cells were then postincubated at 37° C. for another 72 h. Cell viability was determined by a resazurin blue assay, where 10 μL of 60 μM resazurin was added to the plates at 3 h prior to measuring fluorescence (ex: 560, em: 590).

### Example 5

### Characteristics of ELP-GA Molecules

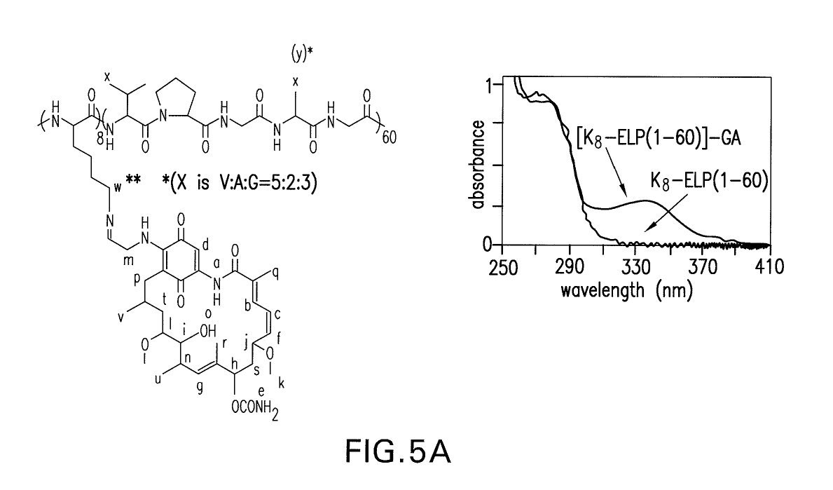

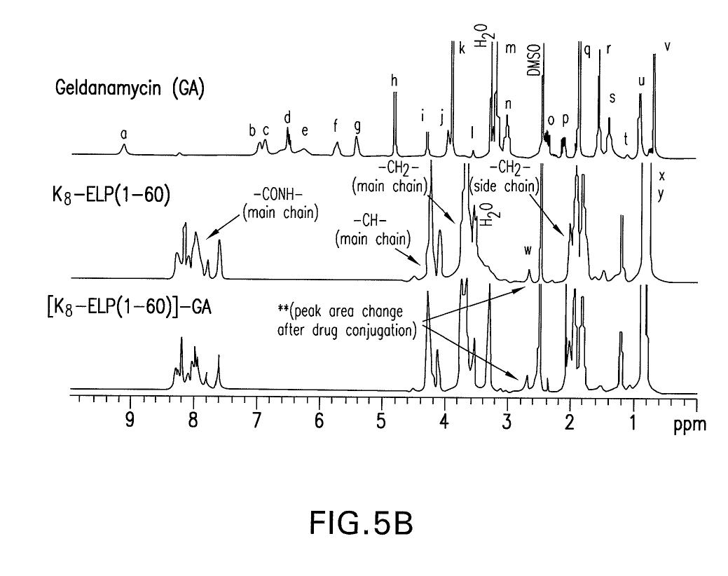

K8-ELP(1-60) block copolymers were biosynthesized by a modified recombinant DNA cloning technique called ‘recursive directional ligation (RDL)’. Short gene segments encoding eight lysine peptides and 10 VPGXG pentapeptides with guest residues Val, Ala and Gly in a 5:2:3 ratio were constructed from chemically synthesized oligonucleotides. These segments were cloned into pUC19 and oligomerized to prepare genes encoding K8-ELP(1-60) and then sub-cloned into a modified pET25b expression vector. The K8-ELP(1-60) was expressed in the E. coli BLR(DE3) strain. Prepared K8-ELP(1-60) diblock copolymers were purified from the E. coli lysate by ITC (1: resuspended cells; 2: cell lysate; 3: soluble proteins after insoluble debris and DNA have been removed; 4: remaining proteins after first hot spin; 5: resuspended product after first round of ITC; 6: remaining proteins after 2nd hot spin; 7: product; 8: ladder). The molecular weight was 25,317 Da, which was determined by MALDI-TOF mass spectrometry. The expected value was 25,445 Da, and the difference with the empirical value would be attributed to the removal of the N-terminal methionine by methionyl-aminopeptidase. Therefore, it is confirmed that K8-ELP(1-60) block copolymers were successfully biosynthesized with high purity. Drug conjugation ratios of [K8-ELP(1-60)]-GA were confirmed by UV spectrometry and ¹H-NMR analysis. As shown in FIG. 5 (inset), [K8-ELP(1-60)]-GA conjugates show an increase in UV absorbance at 337 nm, corresponding to the characteristic spectra of GA derivatives. The drug loading contents were determined based on a calibration curve of GA derivatives, revealing 1.19 and 1.26 molecules of GA(CHO) and GA(OH), respectively, were conjugated to a single K8-ELP(1-60) block copolymer chain. Drug conjugation was also confirmed by a decrease in ¹H-NMR peak areas for methylene groups of the lysines (FIG. 5). Drug loading contents can be derived from a change in ¹H-NMR peak areas, which correspond well with the values calculated by UV spectrometry. It is surprising that the drug conjugation ratio was extremely low <10%, irrespective of the types of derivatives, with respect to the total number of amino groups including the N-terminal and oligolysine (K8) amines of the K8-ELP(1-60) block copolymers. It is suspected that lysine blocks are sterically hindered by the three-dimensional conformation of ELP(1-60) as asserted to the high solubility in the DMSO reaction solvent of ELP(1-60) and K8-ELP(1-60) block copolymers. In addition, steric hindrance may not be due to the physicochemical nature of K8-ELP(1-60) block copolymers, on the contrary, steric hindrance seems to occur during the drug conjugation hampering further drug binding. This hypothesis may require further analysis of intra- and intermolecular structural changes of K8-ELP(1-60) block copolymers and their drug conjugates.

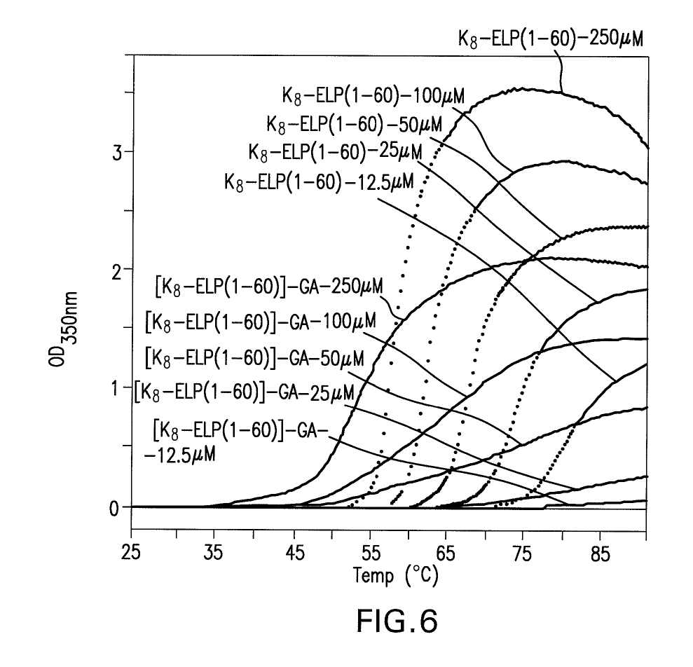

FIG. 6 shows thermosensitive phase transition profiles of K8-ELP(1-60) block copolymers and [K8-ELP(1-60)]-GA(CHO) conjugates. The transition temperature (Tt) is determined as the temperature at which the optical density reaches 50% of the maximum value at 350 nm. The Tt values of K8-ELP(1-60) block copolymers and [K8-ELP(1-60)]-GA(CHO) conjugates were 74.06° C. and 85.54° C., respectively. K8-ELP(1-60) block copolymers underwent a relatively broad phase transition over ˜10° C. This decay in the rapid putative reaction kinetics of ELP may be due to charge-changes repulsions between cationic lysine blocks thereby suppressing intermolecular interactions. Interestingly, the thermosensitive phase transition of [K8-ELP(1-60)]-GA(CHO) conjugates occurs at a lower and broader temperature range while Tt becomes higher. However, when the polymer concentration increased, the Tt of [K8-ELP(1-60)]-GA(CHO) conjugates decreased significantly and eventually became lower than that of K8-ELP(1-60) block copolymers. There is no significant change in such thermosensitive phase transition profiles between GA(CHO) and GA(OH) derivatives. It is generally known that the Tt of ELP changes as a function of concentration, indicating intermolecular interactions play an important role in the observed sharp thermosensitive phase transitions. A broad phase transition display with [K8-ELP(1-60)]-GA conjugates suggests a mixed population; however, it is of interest that the drug conjugation not only reduced the Tt of the conjugates but also broadened the temperature range for phase transition. It is considered that the cationic lysine block can suppress intermolecular interactions at low concentration, yet hydrophobic drug conjugation enhances aggregation between polymer chains significantly at high concentration. Namely, it is obvious that an intramolecular balance between cationic and hydrophobic properties plays a pivotal role in inducing a thermosensitive phase transition of [K8-ELP(1-60)]-GA conjugates, and thus a concentration dependent change in Tt becomes more significant. These results suggest that [K8-ELP(1-60)]-GA conjugates may accumulate in tumor tissues more efficiently as accumulation through the EPR effects enhances in vivo by exploiting characteristic thermosensitive phase transition profiles with Tt that significantly decreases depending on concentration.

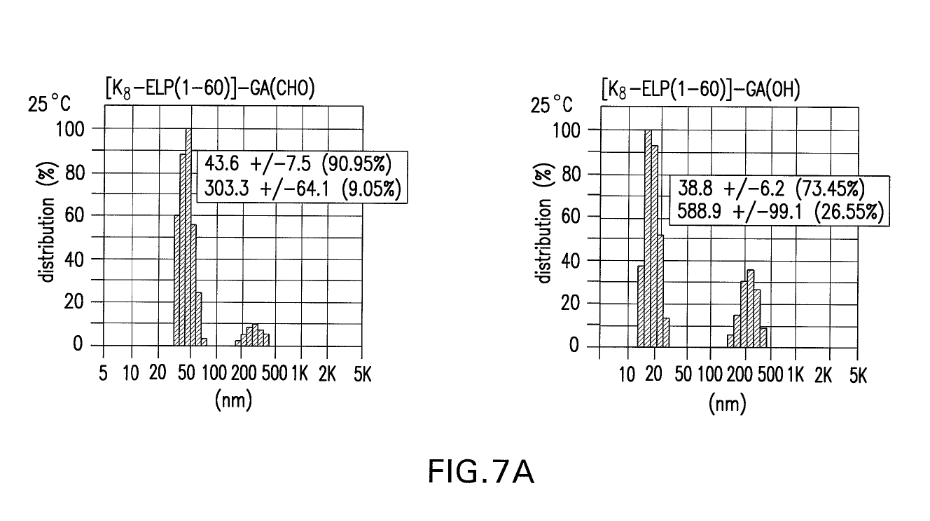

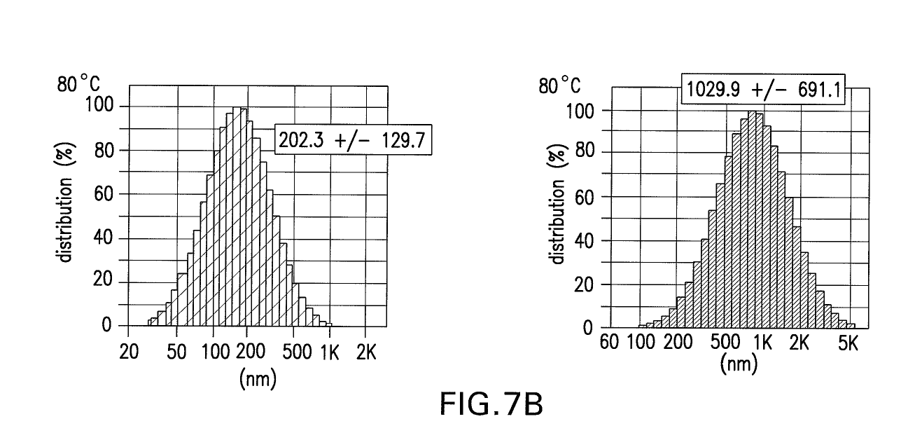

Thermosensitive phase transition profiles display [K8-ELP(1-60)]-GA conjugates have mixed populations. In order to investigate detailed thermosensitive properties of [K8-ELP(1-60)]-GA conjugates, dynamic light scattering (DLS) measurements were completed. Measurement temperatures varied from 25 to 80° C. to observe the difference in size accompanying a phase transition. As shown in FIG. 7, [K8-ELP(1-60)]-GA conjugates prepare nanoparticles with a diameter size less than 50 nm. Yet DLS measurements also show that both [K8-ELP(1-60)]-GA(CHO) and [K8-ELP(1-60)]-GA(OH) conjugates have mixed populations. This finding explains why [K8-ELP(1-60)]-GA conjugates underwent broad thermosensitive phase transitions. Interestingly, however, the [K8-ELP(1-60)]-GA(CHO) prepared nanoparticles with a more unimodal distribution than [K8-ELP(1-60)]-GA(OH). When the temperature increased to 80° C., the particle size increased and small particles disappeared. It is of particularly interest that the particles from [K8-ELP(1-60)]-GA(CHO) conjugates remain relatively small with a ˜202.3 nm size while [K8-ELP(1-60)]-GA(OH) conjugates aggregate to form micron-sized particles (˜1029.9 nm). It is postulated that the intermolecular interaction of [K8-ELP(1-60)]-GA(OH) conjugates increased due to the enhanced hydrophobicity of drug-binding linkers compared to [K8-ELP(1-60)]-GA(CHO) conjugates, and thereby forming large aggregates. These results clearly demonstrate that drug conjugation methods affect the thermosensitive phase transition profile of [K8-ELP(1-60)]-GA conjugates, similar in scope to ELP-anticancer drug conjugates reported by Furgeson et al. and Dreher et al. [18, 20], yet the particles remain in a nanometric order size while undergoing inverse phase transition. Distinct hydrophobicity of [K8-ELP(1-60)]-GA conjugates might be attributed to the difference between the drug binding linkers for GA(CHO) and GA(OH). As shown in FIG. 4, GA(CHO) is conjugated to the lysine moieties of K8-ELP(1-60) directly while GA(OH) is bound to the polymers via a four methylene group spacer plus ester bond. Although drug loading contents are approximately the same, such a chemical difference might affect intermolecular interactions between [K8-ELP(1-60)]-GA conjugates and consequently the aggregation profile. It is not certain which conformation change, between nano- and microsize thermosensitive aggregations, induces higher accumulation in tumor tissue while reducing systemic toxicity in vivo. Nevertheless, it is confirmed that [K8-ELP(1-60)]-GA conjugates form nanoparticles whose particle size can be controlled by heat while stably dispersing in an aqueous solution without precipitation. Such properties are promising for in vivo applications, selecting an intravenous injection as an administration route.

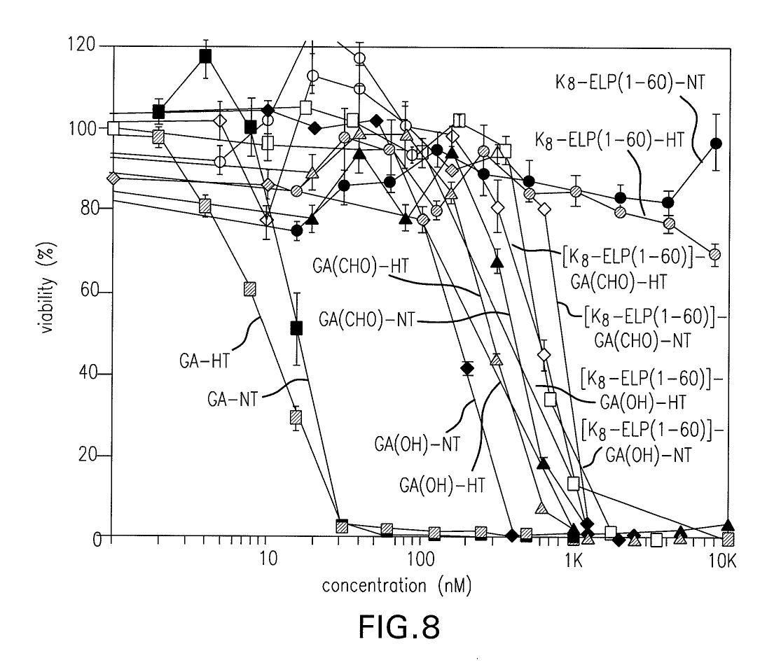

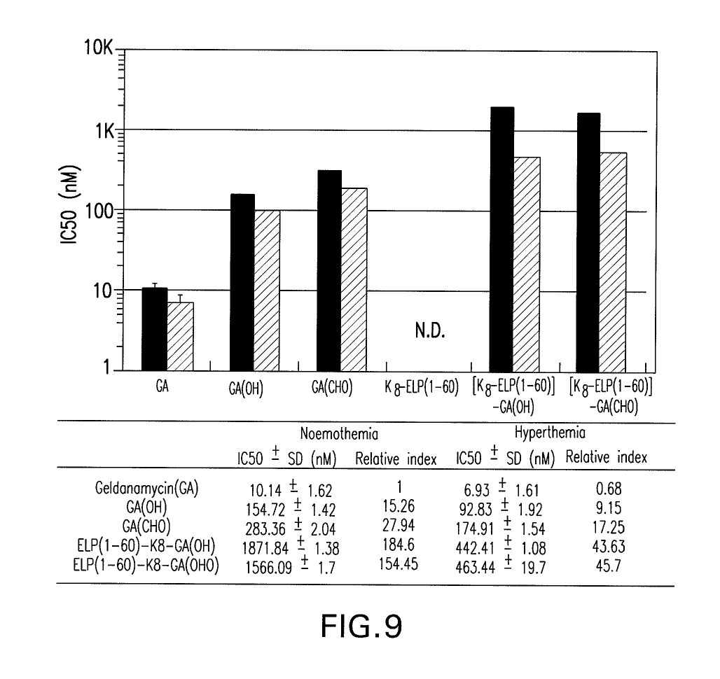

Although [K8-ELP(1-60)]-GA conjugates underwent broad thermosensitive phase transitions, the importance of assessing cytotoxicity remains with of [K8-ELP(1-60)]-GA conjugates alone and in combination with hyperthermia. Such studies could confirm the relevance of hyperthermic combination therapy using HSP90 inhibitors. We hypothesized that normothermic cells treated with GA would become more sensitive to heat shock by downregulating HSP90, thereby reducing cell viability. FIG. 8 displays a cell viability change in a human breast cancer MCF-7 cell line treated with parent GA, GA derivatives, native K8-ELP(1-60), and [K8-ELP(1-60)]-GA conjugates with varying drug concentrations with respect to parent GA in the presence and absence of heat. For hyperthermic combination treatments, heat was applied to the cells at 43° C. for 30 min. The results show that hypothermic combination use of GA is significantly effective to suppress cell viability irrespective of drug and conjugates composition. GA is an inhibitor to downregulate a function of HSP90 that protects cancer cells against hyperthermic stress. FIG. 9 compares the 50% inhibitory concentrations for cell viability (IC₅₀) values among free drugs and drug conjugates. Interestingly, the data show hyperthermic combination effects are the most prominent in case of [K8-ELP(1-60)]-GA conjugates. Although the absolute IC₅₀ values of the conjugates are higher than either the parent GA or GA derivatives, it is expected that the drug delivery efficiency of macromolecules may compensate for low activity in vivo, often observed in DDS using polymer-drug conjugates. Moreover, a significant difference in cytotoxic activity, with and without heat, suggests that systemic toxicity accompanying chemotherapy could be effectively suppressed by thermal targeting of [K8-ELP(1-60)]-GA conjugates. It is still unclear why hyperthermic combination treatments of [K8-ELP(1-60)]-GA were significantly effective to suppress cellular viability. Increased cellular uptake and/or direct interaction against the cellular membrane due to the thermosensitive phase transition may be contributing factors. Nevertheless, considering that the toxicity of native K8-ELP(1-60) block copolymers remained low under the experimental conditions herein, enhanced cytotoxicity by hyperthermia would be due to the intracellular drug action of [K8-ELP(1-60)]-GA conjugates rather than direct interaction with the cells.

### REFERENCES

The following references, to the extent that they provide exemplary procedural or other details supplementary to those set forth herein, are specifically incorporated herein by reference.
U.S. Pat. No. 4,196,265
U.S. Pat. No. 4,261,989
U.S. Pat. No. 4,554,101
U.S. Pat. No. 4,683,202
U.S. Pat. No. 5,354,855
U.S. Pat. No. 5,466,468
U.S. Pat. No. 5,620,479
U.S. Pat. No. 5,693,762
U.S. Pat. No. 5,702,717
U.S. Pat. No. 5,846,225
U.S. Pat. No. 5,846,233
U.S. Pat. No. 5,861,021
U.S. Pat. No. 5,861,155
U.S. Pat. No. 5,922,013
U.S. Pat. No. 5,928,906
U.S. Pat. No. 6,004,573
U.S. Pat. No. 6,020,192
U.S. Pat. No. 6,054,297
U.S. Pat. No. 6,167,313
U.S. Pat. No. 6,117,949
U.S. Pat. No. 6,201,072
U.S. Pat. No. 6,852,834
U.S. Publn. 20030059944
U.S. Publn. 20030105051
U.S. Publn. 20050251234
U.S. Publn. 20050090732
U.S. application Ser. No. 11/402,63
Ausubel et al., In: Current Protocols in Molecular Biology, John, Wiley & Sons, Inc, New York, 1996.
Beketic-Oreskovic et al., Int. J. Hyperthermia, 13(2):205-214, 1997.
Berzal-Herranz et al., Genes Dev, 6(1):129-134, 1992.
Bidwell and Raucher, Mol. Cancer. Ther., 4(7):1076-1085, 2005.
Braiden et al., Hum. Gene Ther., 11(18):2453-2463, 2000.
Brummelkamp et al., Science, 296(5567):550-553, 2002.
Capaldi et al., Biochem. Biophys. Res. Comm., 74(2):425-433, 1977.
Cech et al., Cell, 27(3 Pt 2):487-496, 1981.
Chowrira et al., J. Biol. Chem., 268:19458-62, 1993.
Chowrira et al., J. Biol. Chem., 269(41):25856-25864, 1994.
DeBoer et al., Antibiot. (Tokyo) 23:442-447, 1970.
Dreher et al., J. Control Release, 91(1-2):31-43, 2003.
Elbashir et al., Genes Dev., 5(2):188-200, 2001.
Forster and Symons, Cell, 49(2):211-220, 1987.
Gerlach et al., Nature (London), 328:802-805, 1987.
Glaze et al., Cancer Chemother. Pharmacol., 56:637-647, 2005
Harlow and Lane, In: Antibodies, a Laboratory Manual, Cold Spring Harbor Laboratory, 139-281, 1988.
Haseloff and Gerlach, Nature, 334(6183):585-591, 1988.
Herrero-Vanrell et al., J. Control Release, 102(1):113-122, 2005.
Hildebrandt et al., Crit. Rev. Oncol. Hematol., 43(1):33-56, 2002.
Johnson et al., In: Biotechnology And Pharmacy, Pezzuto et al. (Eds.), Chapman and Hall, NY, 1993.
Joyce, Nature, 338:217-244, 1989.
Kaneda et al., Science, 243:375-378, 1989.
Kato et al, J. Biol. Chem., 266:3361-3364, 1991.
Kim and Cech, Proc. Natl. Acad. Sci. USA, 84(24):8788-92, 1987.
Kim et al., J. Biomed. Mater Res. A, 70(1):154-158, 2004.
Kopecek, Eur. J. Pharm. Sci., 20(1):1-16, 2003.
Kyte and Doolittle, J. Mol. Biol., 157(1):105-132, 1982.
Lieber and Strauss, Mol. Cell Biol., 15(1):540-551, 1995.
Michel and Westhof, J. Mol. Biol., 216:585-610, 1990.
Palukaitis et al., Virology, 99:145-151, 1979.
Parker et al., Anal. Biochem., 302(1):75-80, 2002.
PCT Appln. WO 03/012052
Perriman et al., Gene, 113:157-163, 1992.
Perrotta and Been, Biochemistry, 31(1):16-21, 1992.
Prody et al., Science, 231:1577-1580, 1986.
Reinhold-Hurek and Shub, Nature, 357:173-176, 1992.
Remington's Pharmaceutical Sciences” 15th Edition, pages 1035-1038 and 1570-1580, 1990.
Sambrook et al., In: Molecular cloning: a laboratory manual, 2^{nd} Ed., Cold Spring Harbor Laboratory Press, Cold Spring Harbor, N.Y., 1989.
Sarver et al., Science, 247:1222-1225, 1990.
Scanlon et al., Proc. Natl. Acad. Sci. USA, 88:10591-10595, 1991.
Schild, Prog. Polymer Sci., 17(2):163-249, 1992.
Sharp and Workman, Adv. Cancer Res., 95:323-348, 2006.
Sioud et al., J. Mol. Biol., 223:831-835, 1992.
Smith et al., Cancer Chemother Pharmacol. 56:126-37, 2005
Sosnik and Cohn, Biomaterials, 26(4):349-357, 2005.
Sui et al., Proc. Natl. Acad. Sci. USA, 99(8):5515-5520, 2002.
Symons, Annu. Rev. Biochem., 61:641-671, 1992.
Symons, Nucl. Acids Res., 9(23):6527-6537, 1981.
Thompson et al., Nature Genet., 9:444-450, 1995.
Urry et al., J. Bioact. Compat. Polym., 6(3):263-282, 1991.
Yamauchi et al., Macromolecules, 37(10):3519-3522, 2004.
Yuan and Altman, Science, 263:1269-1273, 1994.
Yuan et al., Proc. Natl. Acad. Sci. USA, 89:8006-8010, 1992.

1. A drug delivery vehicle comprising a heat shock protein (Hsp) 90 antagonist complexed with an elastin-like repeat.

2. The drug delivery vehicle of claim 1, wherein the elastin-like repeat comprises 10 to 500 repeats of the sequence VPGXG wherein, X is any amino acid except proline.

3. The drug delivery vehicle of claim 2, wherein the elastin-like repeat comprises 50 to 300 repeats.

4. The drug delivery vehicle of claim 3, wherein the elastin-like repeat comprises 80 to 200 repeats.

5. The drug delivery vehicle of claim 2, wherein X is valine, alanine or glycine.

6. The drug delivery vehicle of claim 1, wherein the Hsp90 antagonist is covalently linked to the elastin-like repeat.

7. The drug delivery vehicle of claim 6, wherein the covalent linker is a cleavable linker.

8. The drug delivery vehicle of claim 7, wherein the cleavable linker is a pH cleavable linker, an enzyme cleavable linker, a heat cleavable linker, a radiation cleavable linker or a linker that is cleaved in aqueous solution.

9. The drug delivery vehicle of claim 8, wherein the cleavable linker comprises water cleavable ester.

10. The drug delivery vehicle of claim 8, wherein the cleavable linker is an esterase cleavable linker.

11. The drug delivery vehicle of claim 1, wherein the Hsp90 antagonist is radicicol or a derivative thereof.

12. The drug delivery vehicle of claim 1, wherein the Hsp90 antagonist is a benzoquinoid ansamycin.

13. The drug delivery vehicle of claim 12, wherein the benzoquinoid ansamycin is geldanamycin or a geldanamycin derivative.

14. The drug delivery vehicle of claim 13, wherein the geldanamycin derivative is 17-β-hydroxyethylamino-17-demethoxygeldanamycin.

15. The drug delivery vehicle of claim 1, comprising at least two Hsp90 antagonist molecules in complex an elastin-like repeat.

16. The drug delivery vehicle of claims 1, wherein the elastin-like repeat further comprises a cell targeting sequence, a cell penetrating sequence or a localization signal.

17. The drug delivery vehicle of claims 16, wherein the elastin-like repeat comprises a cell targeting sequence.

18. The drug delivery vehicle of claim 17, wherein the cell targeting sequence is an antibody, a ligand, a cytokine or a chemokine.

19. The drug delivery vehicle of claims 18, wherein the antibody is a single chain antibody.

20. The drug delivery vehicle of claims 18, wherein the antibody is a humanized antibody.

21. The drug delivery vehicle of claim 18, wherein the ligand is vascular endothelial growth factor (VEGF).

22. The drug delivery vehicle of claim 16, wherein the fusion protein further comprises a cell penetrating sequence.

23. The drug delivery vehicle of claim 22, wherein the cell penetrating sequence is from the tat or antennapedia polypeptides.

24. The drug delivery vehicle of claim 16, wherein the fusion protein comprises a localization signal.

25. The drug delivery vehicle of claim 24, wherein the localization signal is a nuclear localization signal or a mitochondrial localization signal.

26. The drug delivery vehicle of claim 1, wherein the drug delivery vehicle has a median thermal transition temperature of less than about 48° C.

27. The drug delivery vehicle of claim 1, wherein thermal transition of the drug delivery vehicle occurs over a range of less than about 10° C.

28. The drug delivery vehicle of claim 1, wherein the diameter of the drug delivery vehicle is less than about 1 μm.

29. The drug delivery vehicle of claim 28, wherein the diameter of the drug delivery vehicle is less than about 500 nm.

30. The drug delivery vehicle of claim 29, wherein the diameter of the drug delivery vehicle is less than about 200 nm.

31. The drug delivery vehicle of claim 1, further comprising a conjugated nanoparticle.

32. The drug delivery vehicle of claim 31, wherein the nanoparticle increases in temperature upon exposure to radio frequency radiation (RF).

33. The drug delivery vehicle of claim 31, wherein the nanoparticle is a metal nanosphere or metal nanoshell.

34. The drug delivery vehicle of claim 1, wherein the elastin-like repeat is conjugated to a nucleotide binding polypeptide.

35. The drug delivery vehicle of claim 34, wherein the elastin-like repeat and nucleotide binding polypeptide conjugate are further defined as fusion protein.

36. The drug delivery vehicle of claim 34, wherein at least 25% of the amino acids in the nucleic acid binding polypeptide are positively charged at neutral pH.

37. The drug delivery vehicle of claim 36, wherein at least 25% of the amino acids in the nucleic acid binding polypeptide are lysine residues.

38. The drug delivery vehicle of claim 37, wherein the nucleic acid binding sequence comprises 4 to 100 repeats of the sequence VKG or the sequence VK.

39. The drug delivery vehicle of claims 35, wherein elastin-like repeat and the nucleic acid binding polypeptide are separated by a spacer region.

40. The drug delivery vehicle of claim 39, wherein the spacer comprises a cleavable linker.

41. The drug delivery vehicle of claims 34, further comprising a nucleic acid bound to the nucleic acid binding polypeptide.

42. The drug delivery vehicle of claims 41, wherein the bound nucleic acid is a therapeutic nucleic acid.

43. The drug delivery vehicle of claims 41, wherein the nucleic acid is a therapeutic nucleic acid.

44. The drug delivery vehicle of claim 41, wherein the nucleic acid is a DNA or RNA.

45. The drug delivery vehicle of claim 44, wherein the DNA is a DNA expression vector.

46. The drug delivery vehicle of claim 44, wherein the RNA is a mRNA, a siRNA or a miRNA.

47. A method for treating a cell proliferative, viral or protein aggregation disease in an animal comprising administering to the animal and effective amount of a drug delivery vehicle according to claim 1.

48. The method of claim 47, wherein the animal is a human.

49. The method of claim 47, wherein the cell proliferative disease is a cancer or an angiogenic disorder.

50. The method of claim 49, wherein the angiogenic disorder is ocular neovascularization, Arterio-venous malformations, coronary restenosis, peripheral vessel restenosis, glomerulonephritis or rheumatoid arthritis.

51. The method of claim 49, wherein the cell proliferative disease is a cancer.

52. The method of claim 51, wherein the cancer is a melanoma, non-small cell lung, small-cell lung, lung, hepatocarcinoma, retinoblastoma, astrocytoma, glioblastoma, gum, tongue, leukemia, neuroblastoma, head, neck, breast, pancreatic, prostate, renal, bone, testicular, ovarian, mesothelioma, cervical, gastrointestinal, lymphoma, brain, colon, sarcoma or bladder cancer.

53. The method of claim 51, wherein the cancer is a tumor.

54. The method of claim 47, wherein the drug delivery vehicle is administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, locally, by inhalation (e.g. aerosol inhalation), by injection, by infusion or by continuous infusion.

55. The method of claim 54, wherein the drug delivery vehicle is administered intraveniously, locally or intratumorally.

56. The method of claim 47, the method further comprising administering a second therapy to the animal before, after or concomitently with the drug delivery vehicle.

57. The method of claim 47, wherein the second therapy is a chemotherapy, radiation therapy, immunotherapy, surgical therapy or a hyperthermia therapy.

58. The method of claim 57, wherein the second therapy is a hyperthermia therapy.

59. The method of claim 58, wherein the hyperthermia therapy decreases the aqueous solubility of the drug delivery vehicle.

60. The method of claim 58, wherein the hyperthermia is applied locally.

61. The method of claims 60, wherein the hyperthermia therapy increases the local temperature of the animal to between about 38° C. and 46° C.

62. The method of claim 60, wherein the drug delivery vehicle is administered systemically and the hyperthermia is administered locally.

63. The method of claims 47, wherein the viral disease is hepatitis B or hepatitis C infection.

64. The method of claim 47, wherein the protein aggregation disease is Huntington's disease, Alzheimer's disease, Parkinson's disease, or a prion disease.


SEQUENCE LISTING (5 sequences)

SEQ ID NO: 1 - protein, 5 aa, Artificial
  Synthetic peptide
VPXXG

SEQ ID NO: 2 - DNA, 33 bp, Artificial
  Synthetic primer
gtgggtaaaaaaaagaaaaaaaaaaagaaaggc

SEQ ID NO: 3 - DNA, 33 bp, Artificial
  Synthetic primer
tttctttttttttttcttttttttacccacgcc

SEQ ID NO: 4 - DNA, 27 bp, Artificial
  Synthetic primer
tatgagcgggccgggctggccgtgata

SEQ ID NO: 5 - DNA, 29 bp, Artificial
  Synthetic primer
agcttatcacggccagcccggcccgctca